(19) United States
(12) Patent Application Publication
(10) Pub. No.: US 2025/0051395 A1
(43) Pub. Date: Feb. 13, 2025

(54) PEPTIDES AND METHODS FOR USE IN TREATING PAIN

(71) Applicant: TAFALGIE THERAPEUTICS, MARSEILLE (FR)

(72) Inventors: STÉPHANE GAILLARD, MARSEILLE (FR); FRANCIS CASTETS, MARSEILLE (FR); ABDELAZIZ MOQRICH, MARSEILLE (FR)

(21) Appl. No.: 18/719,934

(22) PCT Filed: Dec. 19, 2022
(86) PCT No.: PCT/EP2022/086559

§ 371 Date: Jun. 14, 2024

(30) Foreign Application Priority Data
Dec. 17, 2021 (EP) 21306835.6

(51) Int. Cl. C07K 14/00; A61K 38/00; A61K 45/06; A61P 25/02; A61P 25/04

(52) U.S. Cl. CPC C07K 14/001; A61K 45/06; A61P 25/02; A61P 25/04; A61K 38/00

(57) ABSTRACT

The present invention relates to novel peptides, compositions and kits comprising the peptides, for use as active ingredient for preventing or treating pain in a subject in need thereof. Also herein described is an in vitro or ex vivo method for modulating the expression of these peptides.

### FIELD OF THE INVENTION

The present application relates generally to the field of pain management. More particularly, the present invention relates to compositions for preventing and/or treating pain, comprising a peptide fragment of TAFA-4 protein as an active ingredient and methods for preventing and/or treating pain, in particular acute, subacute, or chronic pain originating from neuropathic pain, post-operative pain, or inflammatory pain, or for preventing and/or treating hyperalgesia or allodynia resulting from injury-induced pain.

### BACKGROUND OF THE INVENTION

Pain is commonly classified as acute or chronic. Acute pain is short-lived and essential for the maintenance of our physical integrity, whereas chronic pain persists beyond the normal time of healing and adversely affects well-being. Chronic inflammatory, neuropathic or post-operative pains give rise to long-lasting sensory abnormalities such as hyperalgesia (extreme pain evoked by noxious stimuli) and mechanical allodynia (pain evoked by innocuous mechanical stimuli). These categories of pain differ in terms of etiology and clinical features, but they have several mechanisms in common, including alterations to neuro-immune interactions and neuron sensitization, both peripherally and centrally (Costigan et al., 2009). There is growing evidence to suggest that loss of inhibition may be a key mechanism underlying chronic pain (Bourane et al., 2015a; Bourane et al., 2015b; Boyle et al., 2019; Coull et al., 2005; Duan et al., 2014; Peirs et al., 2015; Petitjean et al., 2019; Petitjean et al., 2015; Zeilhofer et al., 2012; Zhang et al., 2018). However, despite our extensive knowledge of the mechanisms and circuits underlying chronic pain in rodents, the translation of these findings into effective treatments for chronic pain in humans remains unsatisfactory (Colloca et al., 2017). Indeed, non-steroidal anti-inflammatory agents (NSAIDs) have limited efficacy against chronic pain, and opioids have multiple adverse effects, including potentially lethal respiratory depression, nausea, constipation, hyperalgesia, tolerance, physical and psychological dependence (Benyamin et al., 2008). Thus, efforts to identify new targets with analgesic or antalgic potential for the treatment of chronic pain should be encouraged.

In the last few years, inventors have discovered striking features of the secreted protein TAFA-4 suggesting that it might be an interesting drug for the treatment of chronic pain (WO2014180853). TAFA-4 belongs to a family of five highly conserved secreted neurokines (Sarver et al., 2021). TAFA-4 contains a signal peptide followed by a highly conserved core region with 10 cysteine residues including a CC-chemokine motif that make it resemble a cytokine (Tom Tang et al., 2004).

Inventors have also previously shown that in mice in which the TAFA-4 gene is deleted, mechanical hypersensitivity induced by nerve injury lasts much longer than in wild-type (WT) mice (Delfini et al., 2013). These phenotypes are reversed by intrathecal injection of recombinant TAFA-4.

As described in WO2014180853, inventors previously reported experimental evidence demonstrating that TAFA-4 possesses powerful actions against pathologically increased mechanical pain. It has been shown that, in both male and female mice, the intrathecal and subcutaneous administration of human recombinant TAFA-4 reverses inflammatory, post-operative and nerve injury-induced mechanical hypersensitivity. They have also shown that TAFA-4 can reverse nerve injury-induced neuronal sensitization of the spinal lamina II interneurons reported to be responsible for mechanical threshold alterations.

Production of recombinant mature TAFA-4 protein may, however, be challenging because TAFA-4 is a cysteine-rich protein (10 cysteines in total). Indeed, refolding can result in obtaining non-native conformations or an improper disulfide bridging pattern which may greatly impact the activity of the protein. Moreover, the several purification steps required for obtaining recombinant TAFA-4 protein with high degree of purity, together with possible aggregation problems, may limit the yield of properly folded protein. Furthermore, while chemical synthesis of TAFA-4 protein was implemented, it is also challenging due to the length of the protein, requiring assembly of not less than 4 (protected or partially protected) peptide segments (starting from the N terminal and ending the most C-terminal peptide). To general a full-length TAFA-4, three ligation purification steps are required, leading to low yields that are not compatible with industrial scale production. Thus, there would be substantial advantage in identifying novel compounds having similar activity as that of TAFA-4 protein, that can be manufactured on an industrial scale and in a cost-effective way.

Thus, there is a strong need for alternative therapeutic agents and methods for use for efficiently preventing or treating pain, in particular acute, subacute or chronic pain.

### SUMMARY OF THE INVENTION

The present invention is based, at least in part, on the identification of a novel peptide (also herein identified as “TT1”) and variants thereof, such as the herein identified “TT6” peptide, having antalgic or analgesic activity, in particular an antalgic activity. The peptide of interest is, or derived from, a specific fragment of the human TAFA-4 protein. Advantageously, this peptide and variants thereof are easy to produce and are highly efficacious against pain, in particular acute, subacute or chronic pain, typically against injury-induced pain.

The present invention relates to an isolated, synthetic or recombinant peptide of sequence SEQ ID NO: 1, or a peptide having at least 90% identity to SEQ ID NO: 1 (also herein identified as “peptide variant” or “variant of the peptide of sequence SEQ ID NO: 1”). In a particular aspect, the peptide of sequence SEQ ID NO: 1, or the variant thereof, is a recombinant peptide. In a particular aspect, the peptide variant is an isolated, synthetic or recombinant peptide of sequence SEQ ID NO:5.

Preferably, similarly to the peptide of SEQ ID NO: 1, the peptide variant having at least 90% identity to SEQ ID NO: 1, such as for example the peptide of SEQ ID NO: 5 (“TT6”), modulates excitability of spinal cord interneurons (preferably spinal cord lamina IIi interneurons). In a preferred aspect, the amino acid residues glutamine (Q) at position 13 and tyrosine (Y) at position 45, with reference to positions set forth in SEQ ID NO: 1, remain unchanged in the peptide variant of the peptide of SEQ ID NO: 1.

The present invention also relates to a nucleic acid encoding a peptide or a variant thereof as herein described; a vector permitting the expression of a peptide or peptide variant as herein above described; and to a cell comprising a nucleic acid encoding such a peptide or variant thereof, or a cell modified using a vector of the invention.

In another aspect, the present invention relates to the peptide herein described, or a variant thereof, for use as an active ingredient, for example as a drug or medicament. It also relates to a composition, typically a therapeutic, veterinary or dietarily composition, comprising a peptide as herein described, or a variant thereof, a nucleic acid encoding such a peptide or variant thereof, and/or a vector or a cell as herein described, and a pharmaceutically and/or dietarily acceptable support.

In this particular aspect, the composition may further comprise at least one additional (distinct) active compound, preferably an active agent efficient against pain (acute, subacute or chronic pain), even more preferably a steroidal anti-inflammatory drug (SAID), a non-steroidal anti-inflammatory drug (NSAID) or an opioid drug.

A further object of the present invention relates to a peptide, a nucleic acid, a vector, or a cell as herein described for use as an active ingredient for preventing or treating pain in a subject in need thereof. It also relates to the use of a peptide, a nucleic acid, a vector, a cell, or a composition as herein described for the manufacture of a medicament for preventing or treating pain in a subject in need thereof. In particular, the invention may be used to treat a chronic pain, a neuropathic pain, a post-operative pain, an inflammatory pain, hyperalgesia or allodynia.

The present invention also relates to a method of preventing or treating pain in a subject in need thereof, comprising administering an (therapeutic) effective amount of a composition as herein described or of the peptide of SEQ ID NO: 1, or a variant thereof having at least 90% identity to SEQ ID NO: 1 such as the peptide of SEQ ID NO: 5.

The peptides or the compositions as herein described may be administered to the subject by any route, such as for example intramuscularly, intravenously, intraperitoneally, orally (per os), anally, cutaneously, subcutaneously, dermically, transdermically or intrathecally. Preferably the peptides or the compositions herein described are administered to the subject subcutaneously or orally, even more preferably orally.

The peptide or the composition described herein may be part of a kit. Thus, the present invention also relates to a kit comprising i) a peptide, a nucleic acid, a vector, a cell or a composition as herein described, ii) at least one additional active compound efficient against pain, preferably distinct from a peptide as herein described or variant thereof, and optionally iii) written instructions for using the kit. The kit herein described is typically used in the context of the prevention or treatment of pain.

In a particular aspect, the products of the present invention may also be used in the context of research. The nucleic acid encoding a peptide or variant as herein described, the vector permitting its expression, or the cell comprising such a nucleic acid or modified using such a vector may be used to express or modulate in vitro or ex vivo the expression of a peptide of SEQ ID NO:1 or a functional variant thereof having at least 90% identity to SEQ ID NO: 1 such as the peptide of SEQ ID NO: 5. In another particular aspect, the present invention also relates to a transgenic animal modified to express a nucleic acid as herein described.

### BRIEF DESCRIPTION OF THE FIGURES

FIG. 1: Antalgic effect of peptide of SEQ ID NO: 1 administered subcutaneously in vivo in the Spared Nerve Injury (SNI) model of neuropathic pain. The figure represents the mechanical force required (expressed in its equivalent in weight in gram) to elicit a paw withdrawal response in 50% of mice in function of time. The peptide of SEQ ID NO: 1 (referred as “Peptide”) is compared to a negative control (vehicle) and a positive control (Pregabalin). Statistical differences between peptide treated mice compared to vehicle group are shown (Two-way RM ANOVA, followed by Bonferroni post-hoc test: *p<0.05, **p<0.01, ***p<0.001).

FIG. 2: Antalgic effect of peptide of SEQ ID NO: 1 administered per os in vivo in an SNI model of neuropathic pain. The figure represents the mechanical force required (expressed in its equivalent in weight in gram) to elicit a paw withdrawal response in 50% of mice in function of time. The peptide of SEQ ID NO: 1 (referred as “Peptide”) is compared to a negative control (vehicle) and a positive control (Pregabalin). Statistical differences between peptide treated mice compared to vehicle group are shown (Two-way RM ANOVA, followed by Bonferroni post-hoc test: *p<0.05, **p<0.01, ***p<0.001).

FIG. 3: Dose-dependent antalgic effect of peptide of SEQ ID NO: 1 administered per os in vivo in a SNI model of neuropathic pain. The data are represented as percentage response to baseline levels. The peptide of SEQ ID NO: 1 (referred as “Peptide”) is compared to a negative control (vehicle) and a positive control (Pregabalin). The measures were performed at 1, 2, 4 and 24 hours post oral gavage. Statistical differences between peptide treated mice compared to vehicle group are indicated in the table (Two-way RM ANOVA, followed by Bonferroni post-hoc test: *p<0.05, **p<0.01, ***p<0.001).

FIG. 4: Comparison of the antalgic effect of TAFA-4 (full length protein) with peptide of SEQ ID NO: 1 administered subcutaneously or per os in a SNI model of neuropathic pain. The figure represents the mechanical force required (expressed in its equivalent in weight in gram) to elicit a paw withdrawal response in 50% of mice in function of time. The peptide of SEQ ID NO: 1 (referred as “Peptide”) is compared to a negative control (vehicle) and to the TAFA-4 protein (full length) (referred as (TAFA4 s.c). Statistical differences between peptide or TAFA4 treated mice compared to vehicle group are shown (Two-way RM ANOVA, followed by Bonferroni post-hoc test: *p<0.05, **p<0.01, ***p<0.001).

FIG. 5: Antalgic effect of the TAFA-4 (full length) protein administered per os in vivo in a SNI model of neuropathic pain. The figure represents the mechanical force required (expressed in its equivalent in weight in gram) to elicit a paw withdrawal response in 50% of mice in function of time. The TAFA-4 (full length) protein is compared to a negative control (vehicle) and to a positive control (pregabalin). Statistical differences between Pregabalin treated mice compared to vehicle group are shown (Two-way RM ANOVA, followed by Bonferroni post-hoc test: *p<0.05, **p<0.01, ***p<0.001). No statistical differences are detected for per os administration of TAFA4.

FIG. 6: Antalgic effect of peptide of SEQ ID NO: 1 administered per os in vivo in an inflammatory pain model (carrageenan model). The figure represents the mechanical force required (expressed in its equivalent in weight in gram) to elicit a paw withdrawal response in 50% of mice in function of time. The peptide of SEQ ID NO: 1 (referred as “Peptide”) is compared to a negative control (vehicle) and a positive control (Celecoxib). Statistical differences between peptide or celecoxib treated mice compared to vehicle group are shown (Two-way RM ANOVA, followed by Bonferroni post-hoc test: *p<0.05, **p<0.01 ***p<0.001).

FIG. 7: Antalgic effect of peptide of SEQ ID NO: 1 administered per os in vivo in a post-operative pain model (Brennan paw incision model of pain). The figure represents the mechanical force required (expressed in its equivalent in weight in gram) to elicit a paw withdrawal response in 50% of mice in function of time. The peptide of SEQ ID NO: 1 (referred as “Peptide”) is compared to a negative control (vehicle) and a positive control (Morphine). Statistical differences between peptide or morphine treated mice compared to vehicle group are shown (Two-way RM ANOVA, followed by Bonferroni post-hoc test: *p<0.05, **p<0.01, ***p<0.001).

FIG. 8: Comparison of the pain relief efficacy of recombinant TT1 (bio-produced) and TT1 obtained by chemical synthesis administered per os in a SNI model of neuropathic pain.

The figure represents the mechanical force required (expressed in its equivalent in weight in gram) to elicit a paw withdrawal response in 50% of mice in function of time. The pain relief efficacy of the peptide of SEQ ID NO:1 produced by chemical synthesis (“TT1 synth”) is compared to that of the peptide of SEQ ID NO:1 (bio)produced in bacteria (“TT1 prod Bact”). Statistical differences between “TT1 synth” or “TT1 prod bact” treated mice compared to vehicle groups are shown (Two-way RM ANOVA, followed by Bonferroni post-hoc test: *p<0.05, **p<0.01, ***p<0.001). n=8; Model=SNI; Sex: males; Administration: per os; Concentration TT1 synth: 300 μg/kg, concentration TT1 prod bact: 300 μg/kg.

FIG. 9: Comparison of the pain relief efficacy between bio-produced TT1 and TT6 administered per os or subcutaneously in a SNI model of neuropathic pain.

The figure represents the mechanical force required (expressed in its equivalent in weight in gram) to elicit a paw withdrawal response in 50% of mice in function of time. The peptide of SEQ ID NO: 1 (“TT1”) is compared to the peptide of SEQ ID NO:5 (“TT6”) each peptide being administered either subcutaneously (A) or per os (B). Statistical differences between TT1 or TT6 treated mice compared to vehicle group are shown (Two-way RM ANOVA, followed by Bonferroni post-hoc test: *p<0.05, **p<0.01, ***p<0.001). n=8; Model=SNI; Sex: males; Administration: per os or subcutaneously; Concentration TT1: 300 μg/kg, concentration TT6: 300 μg/kg.

FIG. 10: Preventive antalgic effect of peptide of SEQ ID NO: 1 administered subcutaneously in vivo in a postoperative pain model (Brennan paw incision model)

A. Schematic representation of the protocol. TT1 or vehicle were administered subcutaneously twice a day at different time point: the day before surgery (D-1), 1 hour before and 1 hour after surgery (and wake-up) at D day, and at D+1 and D+2. Mechanical threshold response measurements at D+1 and D+2 were performed before administration of peptide of SEQ ID NO:1 (“TT1”).

B. The figure represents the mechanical force required (expressed in its equivalent in weight in gram) to elicit a paw withdrawal response in 50% of mice in function of time. The peptide of SEQ ID NO: 1 (referred as “TT1”) is compared to a negative control (vehicle). The data are represented as percentage response to baseline levels. Statistical differences between peptide treated mice compared to vehicle group are shown (Two-way RM ANOVA, followed by Bonferroni post-hoc test: *p<0.05, **p<0.01, ***p<0.001). n=23 for TT1, n=22 for vehicle; Model=postoperative pain model; Sex: 11 males and 12 females for TT1-treated mice, 11 males and 11 females for vehicle group; Administration: subcutaneously; Concentration TT1: 300 μg/kg.

FIG. 11: Absence of tolerance after repeated per os administration of peptide of SEQ ID NO:1 on Chronic Constriction Injury (CCI) neuropathic pain model

After establishment of CCI neuropathic pain model, mice were treated with peptide of SEQ ID NO: 1 (“TT1”) or vehicle for 14 consecutive days starting 10 days after surgery. Antalgic effect of TT1 was determined every two days. The figure represents the mechanical force required (expressed in its equivalent in weight in gram) to elicit a paw withdrawal response in 50% of mice in function of time. The peptide of SEQ ID NO: 1 (referred as “TT1”) is compared to a negative control (vehicle). Statistical differences between peptide treated mice compared to vehicle group are shown (Two-way RM ANOVA, followed by Bonferroni post-hoc test: *p<0.05, **p<0.01, ***p<0.001). Model=CCI neuropathic pain model; Sex: 10 males for TT1-treated mice, n=5 for vehicle group; Administration: per os; Concentration TT1: 300 μg/kg.

### DETAILED DESCRIPTION OF THE INVENTION

### Definitions

The terms used in this specification generally have their ordinary meanings in the art within the context of this invention and the specific context where each term is used. Certain terms are discussed below, or elsewhere in the specification, to provide additional guidance to the skilled reader in describing the methods of the invention and how to use them. Moreover, it will be appreciated that the same item can be said in more than one way. Consequently, alternative language and synonyms may be used for any one or more of the terms discussed herein. Synonyms for certain terms are provided. A recital of one or more synonyms does not exclude the use of the other synonyms.

The terms “peptide”, “peptide variant”, “protein fragment”, “protein segment” are used interchangeably to refer to a polymer of amino acid residues. Such polymers of amino acid residues may contain natural or non-natural amino acid residues. The terms also include post-expression modifications of the peptide, for example, glycosylation, sialylation, acetylation, phosphorylation, carbamethylation and the like. Furthermore, in the context of the present invention, a “peptide” refers to a protein fragment or segment which includes modifications such as deletions, additions, and substitutions (generally conservative in nature), to the native (wild-type) sequence, as long as the peptide maintains the desired activity, i.e. preventing or treating pain. These modifications are preferably deliberate mutations, e.g. are obtained through site-directed mutagenesis.

The terms “isolated peptide” refers to a peptide that is removed from its original environment (i.e. the natural environment, if it is naturally occurring). A peptide naturally present in a natural system, for example in a living animal, is to be distinguished from the same peptide, which has been separated from all or part of the coexisting materials in said natural system. The separated peptide is herein designated as an “isolated peptide”. In other words, in the context of the present invention the isolated peptide is a fragment of the TAFA-4 protein which is absent as such from nature.

The terms “synthetic peptide” refer to a peptide obtained by chemical synthesis.

The terms “recombinant peptide” refer to a peptide encoded by recombinant DNA, which has been cloned in a foreign expression system to support the expression of the exogenous gene. The recombinant DNA, usually the cDNA sequence of the target peptide, is typically designed to be placed under the control of a promoter allowing the expression of the target peptide within the chosen host cell. A person of ordinary skill in the art is able to select an appropriate promoter in order to achieve a high-level of protein expression.

The terms “sequence identity”, “sequence having at least X % identity” and “sequence X % identical to” are used interchangeably to refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Preferably, sequence identity is determined over the entire length of a reference sequence, here SEQ ID NO: 1 (“CFPGQVAGTTRAQPSCVEASIVIQKWWCHMNPCLEGEDCKVLPDYSGWSCSSGNKVKT TKVTR”). Considering the particular example of SEQ ID NO: 5 (“SFPGQVAGTTRAQPSSVEASIVIQKWWSHMNPSLEGEDSKVLPDYSGWSSSSGNKVKTT KVTR”), said sequence is more than 90% identical to SEQ ID NO: 1, concretely it is 90.48% identical to SEQ ID NO: 1 using BLAST algorithm.

Optimal alignment of sequences for comparison can be conducted, e.g., by the local homology algorithm of Smith and Waterman, (1981) Adv. Appl. Math. 2:482, by the homology alignment algorithm of Needleman and Wunsch, (1970) J. Mol. Biol. 48:443, by the search for similarity method of Pearson and Lipman, (1988) Proc. Natl. Acad. Sci. USA 85:2444, by computerized implementations of these algorithms (GAP, BESTFIT, FASTA, and TFASTA), or by visual inspection (see generally, Current Protocols in Molecular Biology, F. M. Ausubel et al., eds., Current Protocols, Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al., (1990), J. Mol. Biol. 215: 403-410 and Altschul et al., (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T” when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (“E”) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, (1989) Proc. Natl. Acad. Sci. USA 89:10915).

The degree of percent amino acid sequence identity can also be obtained by ClustalW analysis (version W 1.8) by counting the number of identical matches in the alignment and dividing such number of identical matches by the length of the reference sequence, and using the following default ClustalW parameters to achieve slow/accurate pairwise optimal alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

The terms “nucleic acid” or “polynucleotide” as used herein refer to a polymeric form of nucleotides of any length, either ribonucleotides or deoxyribonucleotides. Thus, these terms include, but are not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The term “vector” refers to a DNA or RNA molecule that comprises a polynucleotide sequence that encodes a peptide, a polypeptide or a protein. A vector generally contains regulatory elements capable of directing expression of the encoding polynucleotide sequence, also called transgene, in the cells into which the nucleic acid molecule is introduced. The term “transgene” refers to a polynucleotide that is introduced into a cell and is capable of being transcribed into RNA and optionally, translated and/or expressed under appropriate conditions. In certain aspects, it confers a desired property to a cell into which it was introduced, or otherwise leads to a desired technical effect, here typically a therapeutic effect. A transgene may contain sequence coding for one or more proteins or one or more fragments of proteins.

The term “gene therapy” refers to the treatment of a subject which involves delivery of a gene/nucleic acid into the cells of an individual for the purpose of preventing or treating a disease.

The term “transfection” refers to the uptake of a foreign polynucleotide by a cell, such as a prokaryote or eukaryote cell. A cell is identified as “transfected” when exogenous polynucleotide has been introduced into the cell. A number of transfection techniques are generally known in the art. See, e.g., Graham et al., Virology 52:456 (1973), Sambrook et al, Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids into suitable host cells.

The term “transduction” refers to the delivery of a nucleic acid molecule into a recipient host cell, such as by a gene delivery vector, for example a recombinant viral vector, in particular a retrovirus, an adenovirus, a recombinant adeno-associated virus (AAV), a herpes simplex virus and a lentivirus. For example, transduction of a target cell by a rAAV virion leads to transfer of the rAAV vector contained in that virion into the transduced cell.

A recombinant “adeno-associated virus (AAV)” is a small Dependoparvovirus with a single-stranded linear DNA genome, lacking pathogenicity and presenting low immunogenicity, that has been artificially produced using recombinant methods. Recombinant AAVs (rAAVs) preferably have tissue/cell-specific targeting capabilities, such that a transgene of the rAAV will be delivered specifically or preferentially to one or more predetermined tissue(s)/cell(s). The AAV capsid as well as the type of regulatory region and administration route are important elements in determining these tissue-specific targeting capabilities.

The term “pain”, refers within the context of the present invention to any pain or sensitivity associated with tissue damage. Preferably, the term pain as used therein is understood as an abnormal sensitivity, i.e., typically as a hypersensitivity which is mediated by aberrantly increased activity of all types of sensory neurons including nociceptors and non-nociceptors. The term pain includes any pain selected from a nociceptor-mediated pain (also called herein a “nociceptive pain”), a neuropathic pain, an inflammatory pain, a pathological pain, an acute pain, a subacute pain, a chronic pain, a mechanical pain, a chemical pain, a somatic pain, a visceral pain, a deep somatic pain, a superficial somatic pain, a somatoform pain, allodynia, hyperalgesia, or a pain associated with a nerve injury.

“Nociceptive” pain or “nociceptor-mediated” pain occurs in response to the activation of a specific subset of peripheral sensory neurons (nociceptors) by intense or noxious stimuli. Nociceptive pain according to the invention includes mechanical pain (crushing, tearing, etc.) and chemical (iodine in a cut, chili powder in the eyes). Examples of nociceptive pains include but are not limited to traumatic or surgical pain, labor pain, sprains, bone fractures, burns, bumps, bruises, injections, dental procedures, skin biopsies, and obstructions. Nociceptive pain includes visceral pain and somatic pain, in particular deep somatic pain and superficial somatic pain.

Visceral pain is diffuse, difficult to locate and often refers to a distant, usually superficial, structure. It may be accompanied by nausea and vomiting and may be described as sickening, deep, squeezing, and/or dull. Deep somatic pain is initiated by stimulation of nociceptors in ligaments, tendons, bones, blood vessels, fasciae and muscles, and is a dull, aching and poorly localized pain. Examples of deep somatic pain include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns.

The terms “injury-induced pain” encompass within the context of the present invention neuropathic pain, inflammatory pain and post-operative pain.

Inflammatory pain is a pain that occurs in the presence of tissue damage or inflammation including post-operative, post-traumatic pain, arthritic (rheumatoid or osteoarthritis) pain, pain associated with autoimmune disease (such as psoriasis) and pain associated with damage to joints, muscle and tendons as in axial low back pain. Inflammation is responsible for the sensitization of peripheral sensory neurons, leading to spontaneous pain and invalidating pain hypersensitivity. Acute or chronic pathological tissue inflammation strongly impacts on pain perception by sensitizing peripheral sensory neurons, giving rise to local and incapacitating pain hypersensitivity. Inflammatory mediators are known to enhance nociceptive primary afferent fibers excitability in part by modifying expression and/or function of ion channels present in nerve endings.

Neuropathic pain is a common type of chronic, non-malignant pain, which is the result of an injury or malfunction in the peripheral or central nervous system. Neuropathic pain may have different etiologies, and may occur, for example as a consequence of a trauma, surgery, herniation of an intervertebral disk, spinal cord injury, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs. A particular type of neuropathic pain is a «Chemotherapy-induced peripheral neuropathic pain» (CIPN) or a «Chemotherapy-induced neuropathic pain» (CINP). CINP or CIPN are among the most severe side effects of anticancer agents, such as platinum- and taxane-derived drugs (oxaliplatin, cisplatin, carboplatin and paclitaxel). CINP may even be a factor of interruption of treatment and consequently increases the risk of death. Neuropathic pain is often characterized by or responsible for the appearance of chronic allodynia (defined as pain resulting from a stimulus that does not ordinarily elicit a painful response, such as light touch) and/or hyperalgesia (defined as an increased sensitivity to a normally painful stimulus), and may persist for months or years beyond the apparent healing of any damaged tissues. Pain may also occur in patients with cancer, which may be due to multiple causes such as inflammation, compression, invasion, metastatic spread into bone or other tissues. Pain also includes migraine and a headache associated with the activation of sensory fibers innervating the meninges of the brain. The peptides according to the invention, such as peptides of SEQ ID NO: 1 or SEQ ID NO:5 are used for treating such above-described types of pain.

The peptides of the invention may be used also for treating pain related to Ehlers-Danlos syndrome which comprises chronic muscle and/or bone and/or joint pain.

Preferably, the peptides of the invention are used for preventing or treating injury-induced pain. More preferably, the peptides of the invention are used for preventing or treating a neuropathic pain (such as a chemotherapy-induced neuropathic pain or a chemotherapy-induced peripheral neuropathic pain), a post-operative pain and/or an inflammatory pain. As shown in the experimental part, inventors also demonstrate that the peptides of the invention are suitable to prevent the subject to develop postoperative mechanical allodynia. Advantageously, the peptides of the invention can be used without inducing any tolerance in the subject.

Typically, the peptides of the invention are used for preventing or treating chronic injury-induced pain. Typically, the peptides of the invention are used for preventing or treating chronic neuropathic pain such as chronic chemotherapy-induced peripheral neuropathic pain (CIPN), a chronic chemotherapy-induced neuropathic pain (CINP) or a chronic nerve injury-induced pain; chronic post-operative pain; and/or chronic inflammatory pain.

Within the context of the present invention, the term “treatment” or “treating” pain in a subject, designates delaying, stabilizing, curing, healing, alleviating, relieving, altering, ameliorating, improving, remedying or affecting any form of pain in a subject as described herein, or any disease or condition associated with pain, in particular acute, subacute or chronic pain (in particular any neuropathic condition associated with chronic pain resulting typically from a neuropathic pain, post-operative pain or inflammatory pain), or any symptom of such a disease or condition, after the application or administration of a suitable peptide of sequence SEQ ID NO: 1 or a variant thereof as herein defined, or of a composition according to the invention.

The term “treatment” or “treating” also refers to any indicator of success in the treatment of pain (which may be associated with any injury, pathology or condition), including any objective or subjective parameter such as abatement, remission, slowing progression or severity, stabilization, diminishing of symptoms of pain, or making it tolerable or more tolerable to the subject. The term “treating” pain, also includes increasing pain tolerance and/or decreasing perceived pain. In particular aspects, the methods, compounds and compositions of the invention are for increasing pain tolerance and/or for decreasing perceived pain. As used herein, the terms “pain tolerance” refers to the amount of pain that a subject can perceive and withstand before breaking down emotionally and/or physically. Pain tolerance is distinct from pain threshold (the minimum mechanical stimulus necessary to produce pain). As used herein, “increasing pain tolerance” generally refers to a situation where a subject can develop a greater pain tolerance (that is, less perceived pain) when compared to a previous state, for instance, following administration of suitable peptide of sequence SEQ ID NO: 1 or a variant thereof or of a composition comprising said peptide or variant to a subject.

Within the context of this invention, “preventing” or “prevention” in relation to pain in a subject, refers to at least the reduction of likelihood of the risk of acquiring (or susceptibility to acquire) any kind of pain by a subject, after the application or administration of a suitable peptide of sequence SEQ ID NO: 1 or a variant thereof, or of a composition according to the invention. For example, “preventing” includes causing at least one of the clinical symptoms of pain not to develop in a subject that may be exposed to or predisposed to, but does not yet experience or display symptoms of pain.

In the context of the invention, a “subject” or “patient” designates an animal, in particular a mammal in need of treatment for a disease or disorder or a symptom thereof. The subject may be a subject having been diagnosed as suffering of a disease or disorder, or determined to be at risk of developing a disease or disorder, said disease or disorder being known to cause the subject to feel pain. In a particular example, the subject is diagnosed or suffering from pain, such as acute pain and/or sub-acute pain or chronic pain, including neuropathic pain, post-operative pain, inflammatory pain, hyperalgesia and/or allodynia.

In a particular aspect, the subject is a human-being.

In another particular aspect, the subject is an animal, in particular a domestic or breeding animal, in particular a horse, a dog, a cat, a cow, etc.

In another particular aspect, the subject has at least one mutated allele in the myo1A gene.

### Peptides

The inventors identified novel peptides that can prevent or treat pain, in particular reverse mechanical hypersensitivity in the context of injured or inflamed nervous system. Inventors believe that by modulating excitability of spinal cord network, these peptides exhibit either antalgic or analgesic activity, in particular antalgic activity, by specifically targeting mechanically and/or chemically induced nociceptive signals.

The herein described peptides are/consist of specific fragments from the human TAFA-4 mature protein of 105 amino acid residues (resulting from the cleavage of the signal sequence and identified in the public data base under the accession #NP_0011005527 or under the Genbank accession AAP92409 as disclosed by Tang et al., 2004).

In a first aspect, the present invention relates to an isolated, synthetic or recombinant peptide of sequence SEQ ID NO: 1, or to a peptide having at least 90% identity to SEQ ID NO: 1 such as the variant peptide of SEQ ID NO: 5.

In a particular aspect, the peptide of sequence SEQ ID NO: 1, or the variant thereof such as the variant peptide of SEQ ID NO: 5, is a recombinant peptide (i.e. a bio-produced peptide).

In a particular aspect, a peptide according to the present invention is a peptide comprising the amino acid sequence CFPGQVAGTTRAQPSCVEASIVIQKWWCHMNPCLEGEDCKVLPDYSGWSCS SGNKVKTTKVTR (SEQ ID NO: 1), i.e. Cys-Phe-Pro-Gly-Gln-Val-Ala-Gly-Thr-Thr-Arg-Ala-Gln-Pro-Ser-Cys-Val-Glu-Ala-Ser-Ile-Val-Ile-Gln-Lys-Trp-Trp-Cys-His-Met-Asn-Pro-Cys-Leu-Glu-Gly-Glu-Asp-Cys-Lys-Val-Leu-Pro-Asp-Tyr-Ser-Gly-Trp-Ser-Cys-Ser-Ser-Gly-Asn-Lys-Val-Lys-Thr-Thr-Lys-Val-Thr-Arg, with a molecular weight of 6920 g/mol.

In another particular aspect, a peptide according to the present invention is a peptide consisting, or consisting essentially of, the amino acid sequence set forth in SEQ ID NO: 1.

In a further particular aspect, a peptide according to the present invention is a peptide consisting, or consisting essentially of, the amino acid sequence set forth in SEQ ID NO: 5.

The terms “consisting essentially of” have the meaning generally ascribed to them e.g., they allow for elements not explicitly recited, but exclude elements that are found in the prior art or that affect a basic or novel characteristic of the invention. As such, a peptide “consisting essentially of” a specified sequence refers to peptide comprising or consisting of this sequence and that include other features not essential to the activity of the peptide.

The peptide of SEQ ID NO: 1 consists, or consists essentially, of the 63 C-terminal amino acid residues of the human TAFA-4 mature protein, and may be identified as amino acids 78-140 of UniProt accession #Q96LR4.

The two amino acid residues Q (Glu) at position 13 and Y (Tyr) at position 45 appearing in bold within SEQ ID NO: 1 (CFPGQVAGTTRAQPSCVEASIVIQKWWCHMNPCLEGEDCKVLPDYSGWSCSSGNKVKTT KVTR), with reference to positions set forth in SEQ ID NO: 1, are believed by inventors to be implied in the biological activity of the peptide, in particular in the modulation of the excitability of spinal cord interneurons, preferably spinal cord lamina IIi interneurons.

Variants of peptide of SEQ ID NO: 1 are also herein disclosed for the first time by inventors. Variants are intended to designate a peptide derived from the peptide of SEQ ID NO: 1 and resulting from the deletion or addition of one or more amino acids and/or substitution of one or more amino acids at one or more sites in the peptide of SEQ ID NO: 1. Variants will be considered functional variants if the variant substantially retains the functional activity of the peptide of SEQ ID NO: 1, or even improve said functional activity allowing the prevention or treatment pain. The activity may be measured using functional assays such as behavioral assays as performed in the experimental part.

A variant of the peptide usable in the context of the present invention may have at least 90%, 90.5%, 91%, 92%, 92.1%, 93%, 93.7%, 94%, 95%, 95.2%, 96%, 96.8%, 97%, 98%, 98.4% or 99% sequence identity to SEQ ID NO: 1.

A particular and preferred variant of the peptide of SEQ ID NO: 1 (“TT1”) is the peptide of SEQ ID NO: 5 (“TT6”): SFPGQVAGTTRAQPSSVEASIVIQKWWSHMNPSLEGEDSKVLPDYSGWSSSSGNKVKTTK VTR, i.e. Ser-Phe-Pro-Gly-Gln-Val-Ala-Gly-Thr-Thr-Arg-Ala-Gln-Pro-Ser-Ser-Val-Glu-Ala-Ser-Ile-Val-Ile-Gln-Lys-Trp-Trp-Ser-His-Met-Asn-Pro-Ser-Leu-Glu-Gly-Glu-Asp-Ser-Lys-Val-Leu-Pro-Asp-Tyr-Ser-Gly-Trp-Ser-Ser-Ser-Ser-Gly-Asn-Lys-Val-Lys-Thr-Thr-Lys-Val-Thr-Arg, with a molecular weight of 6 820 g/mol.

In a particular aspect, the variant having at least 90% identity to SEQ ID NO: 1 modulates excitability of spinal cord interneurons (preferably spinal cord lamina IIi interneurons). In a preferred aspect, the variant is a peptide comprising one or more point mutation(s) (e.g. 2, 3, 4, 5 or 6 point mutations) that add, delete or substitute any of the amino acids present in SEQ ID NO: 1, with the proviso that amino acid residues Q at position 13 and Y at position 45 in SEQ ID NO: 1 remain unchanged (positions 13 and 45 being with reference to positions set forth in SEQ ID NO: 1).

Thus, in a preferred aspect of the invention, the two amino acids (Q at position 13 and Y at position 45 of SEQ ID NO: 1) remain unchanged in the amino acid sequence of the variant of the peptide having at least 90% identity to SEQ ID NO: 1, as for the peptide of SEQ ID NO: 5 for example.

In another particular aspect, a variant of the peptide usable in the context of the present invention may have at least 84.1%, 85%, 85.7%, 86%, 87%, 87.3%, 88%, 88.9% or 89% sequence identity to SEQ ID NO: 1. Preferably, the variant having at least 85% identity to SEQ ID NO: 1 modulates excitability of spinal cord interneurons (preferably spinal cord lamina IIi interneurons).

In a particular aspect, the variant is a peptide comprising seven or more point mutations (e.g. 8, 9 or 10 point mutations) that add, delete or substitute any of the amino acids present in SEQ ID NO: 1, with the proviso that the amino acid residues Q at position 13 and Y at position 45 in SEQ ID NO: 1 remained unchanged (positions 13 and 45 being with reference to positions set forth in SEQ ID NO: 1).

In a particular aspect, the one or more deletions are at the N terminus of SEQ ID NO: 1. In another aspect, the one or more deletions are at the C terminus, or in any other position of SEQ ID NO: 1 with the proviso that the two amino acids at position 13 and at position 45 of SEQ ID NO:1 (Q and Y) remain unchanged. In again another aspect, the two or more deletions are at both the N and C terminus of SEQ ID NO: 1. Such deletions at the N and/or C terminus of the peptide of SEQ ID NO: 1 or in the core thereof may result in a truncated human peptide variant of SEQ ID NO: 1.

In addition, the peptide according to the present invention may be fused to another peptide or protein to form a conjugate usable in the methods herein described. The term “conjugate” in this context refers to an engineered fusion construct combining the biological functions of two molecules within a single polypeptide, namely a peptide of SEQ ID NO: 1 or a variant thereof, for example capable of modulating excitability of a nociceptor or an interneuron, together with for example a polypeptide which specifically interacts or binds to a target cell.

The present invention also relates to a nucleic acid (of SEQ ID NO: 2 or 9) encoding a peptide of SEQ ID NO: 1 or a variant thereof as herein described. Any sequence coding either for the peptide of SEQ ID NO: 1 or for a variant thereof are encompassed by the present invention as well as similar sequences resulting from the degeneration of the genetic code. In a particular aspect, the nucleic acid encoding a peptide as herein described comprises, consists or consists essentially of a sequence of

SEQ ID NO: 2:
TGCTTCCCGGGACAGGTGGCGGGCACAACTCGGGCTCAACCTTCTTGTG
TTGAAGCTTCCATTGTGATTCAGAAATGGTGGTGTCACATGAATCCGTG
TTTGGAAGGAGAGGATTGTAAAGTGCTGCCAGATTACTCAGGTTGGTCC
TGTAGCAGTGGCAATAAAGTCAAAACTACGAAGGTAACGCGG.

In another particular aspect, the nucleic acid encoding a peptide as herein described comprises, consists or consists essentially of a sequence of SEQ ID NO: 9: TGCTTTCCAGGTCAAGTTGCGGGAACAACTCGTGCACAACCATCGTGCGTAGAGGCCT CAATTGTTATCCAAAAGTGGTGGTGTCACATGAACCCCTGCCTCGAAGGAGAGGACTG TAAGGTACTGCCTGACTACAGCGGGTGGTCATGTTCATCAGGCAATAAGGTGAAGACG ACCAAAGTTACCCGT. The nucleic acid of SEQ ID NO:9 is particularly adapted for production of the peptide of SEQ ID NO:1 in bacteria.

The present invention also relates to a nucleic acid (of SEQ ID NO: 6) encoding a peptide of SEQ ID NO: 5 or a variant thereof as herein described. Any sequence coding either for the peptide of SEQ ID NO: 5 or for a variant thereof are encompassed by the present invention as well as similar sequences resulting from the degeneration of the genetic code. In a particular aspect, the nucleic acid encoding a peptide as herein described comprises, consists or consists essentially of a sequence of

SEQ ID NO: 6:
TCGTTTCCAGGTCAAGTTGCGGGAACAACTCGTGCACAACCATCGTCGG
TAGAGGCCTCAATTGTTATCCAAAAGTGGTGGTCGCACATGAACCCCTC
GCTCGAAGGAGAGGACTCGAAGGTACTGCCTGACTACAGCGGGTGGTCA
TCGTCATCAGGCAATAAGGTGAAGACGACCAAAGTTACCCGT

The above-mentioned nucleic acid sequence encoding the peptide of SEQ ID NO: 1 or a variant thereof, can be flanked by regulatory sequences for controlling its expression in an appropriate host cell.

In another aspect, the present invention also relates to an expression cassette comprising, in this order from 5′ to 3′:
a promoter,
a nucleic acid sequence encoding a peptide of SEQ ID NO: 1 or a variant thereof as herein described; and
termination signals, such as for example a polyadenylation signal.

The above-mentioned nucleic acid sequence encoding the peptide of SEQ ID NO: 1 or a variant thereof, or the expression cassette disclosed herein can be flanked by sequences suitable for their packaging into a vector that optimizes its transcription and/or translation in a cell.

In another aspect, the present invention also relates to a vector permitting expression of a peptide as herein described.

### Methods for Producing the Peptide

To allow expression in host cells, the nucleic acid encoding a peptide (or one of its variant) as herein described can be present in a vector and, after introduction of said vector into a suitable host cell, the sequence can be expressed to produce the encoded peptide herein described according to standard cloning and expression techniques, which are well-known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). Various expression vectors can be employed to express a polynucleotide sequence encoding a peptide as herein described. An expression vector that can be used in the present invention includes non-exhaustively an eukaryotic expression vector, in particular a mammalian expression vector, a virus-based expression vector, a baculovirus expression vector, a plant expression vector and any plasmid expression vector in order to produce anyone of the herein described peptide in a host cell. The expression vector could also be a vector allowing the expression of the peptide in a bacterial system.

The choice of expression vector depends on the intended host cells in which the vector is to be expressed. This choice will be easily made by the skilled person. The present invention also relates to a cell, in particular a host cell, comprising a nucleic acid sequence encoding a peptide as herein described. A (host) cell modified using a vector as herein described is also herein disclosed for the first time by inventors.

It is possible to express the peptide of the present invention, or variants thereof, in either prokaryotic or eukaryotic host cells. Representative host cells include many E. coli strains, mammalian cell lines, such as for example CHO, CHO-K1, and HEK293; insect cells, such as Sf9 cells; and yeast cells, such as S. cerevisiae and P. pastoris.

The nucleic acid or vector herein described may be transfected into a host cell by standard techniques commonly used for the introduction of exogenous DNA into a prokaryotic or eukaryotic host cell, such as for example electroporation, calcium phosphate precipitation, DEAE-dextran transfection, etc. Alternatively, the nucleic acid or vector herein described may be delivered into a host cell by transduction using a viral-based vector.

Purity may be measured by any appropriate standard method, for example, by column chromatography, thin layer chromatography, or high-performance liquid chromatography (HPLC) analysis. Isolated or synthetic peptide also defines a degree of sterility that is safe for administration to a human subject, e.g., lacking infectious or toxic agents.

The peptides used in the compositions and methods herein described can also be produced by solid phase synthesis technique. Direct chemical synthesis of peptides can be accomplished by methods very well-known by the person skilled in the art, such as Native Chemical Ligation (NCL). This chemical approach consists of coupling of unprotected peptide fragments: peptide with N-terminal cysteine reacts with a C-terminal thioester peptide. This transthioesterification is rapidly followed by an intramolecular S,N-acyl shift that leads to the formation of a native amide bond at the ligation site. When two peptides are ligated in a one-step ligation followed by a one-step purification, the expected yield is very high and thus compatible with large scale production for therapeutics use.

### Gene Therapy

By cloning the nucleic acid sequences encoding the peptides herein described into appropriate vectors, the inventors also herein provide novel gene delivery tools, in particular genetic constructs (such as for example a cassette of expression or a vector) for gene therapy of pain, in particular acute, subacute or chronic pain, preferably chronic pain. Gene therapy may be employed to allow the endogenous production of a TAFA-4-peptide, a TT1 peptide or a variant thereof, such as the TT6 peptide, by specific cells in a subject. Gene therapy can either occur in vivo or ex vivo. Ex vivo gene therapy requires the isolation and purification of at least a sample of subject's cells, the introduction of a nucleic acid sequence (i.e. transgene) encoding a peptide as herein described into the isolated cells, and the introduction of the genetically altered/modified cells back into the subject. In contrast, in in vivo gene therapy, the transgene is typically packaged for administration to the subject. Gene delivery constructs may be either non-viral or viral. Preferably, the genetic construct herein described is prepared with viral elements, viral vectors and/or any viral packaging system(s) that may be used to express a transgene/coding sequence (i.e. a nucleic acid sequence encoding the peptide of SEQ ID NO: 1 or a variant thereof) in a targeted tissue/cell. The viral vectors can incorporate any suitable promoter and other transcription regulator that allow or facilitate expression of the transgene product in the targeted tissue/cell. The viral packaging system is preferably adapted to the targeted cell. Once into the target cell, such a system facilitates the delivery to the targeted tissue. The viral vector usable in the methods herein described is preferably a replication-deficient virus, such as for example an adenovirus or adeno-associated virus (AAV) vector.

In some aspects, the present invention relates to a recombinant adeno-associated virus (AAV) comprising in its genome, a nucleic acid sequence encoding a peptide of SEQ ID NO: 1 or a variant thereof, such as the peptide of SEQ ID NO: 5, which is typically operably linked to a promoter.

To date, at least dozens different serotypes of AAVs with variations in their surface properties have been isolated from human or non-human primates (NHP) and characterized. The term “serotype” allows the skilled person to distinguish between AAV having serologically different capsids. Serologic distinctiveness is determined on the basis of the lack of cross-reactivity between antibodies to one AAV serotype as compared to other AAV serotypes. The rAAV herein described, also named rAAV vector or rAAV particle, may have any one of the following known serotypes, i.e. may be selected for example from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rhIO, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. The rAAV vector may have enhanced tropism for a particular cell, tissue or organ. In the context of an administration by oral route, the rAAV vector may have enhanced tropism for stomach, small intestine or colon tissue and more specifically for cells that constitute these tissues, in particular for epithelial cells, such as for example enterocytes, Goblet cells, enteroendrocine cells, Paneth cells, or Tuft cells. For targeted cells located in, or targeting cells delivered to, the gut, anyone of AAV 4, AAV7, AAV8, AAV9 or AAV10 may be selected as particularly efficient.

Gene therapy vectors or cassettes of the present invention may be produced by methods known in the art well-known by the skilled person and previously described, e.g., in PCT Patent Application Publication No. WO03042397 and U.S. Pat. No. 6,632,670.

The present description also relates to a method for producing a recombinant vector, for example a viral vector such as an AAV, comprising:
a) culturing a cell that has been transfected/transduced with a recombinant vector described herein; and
b) recovering the recombinant vector from the supernatant of the transfected/transduced cells.

Gene therapy vector of the present invention may be produced by the transfection of two or three plasmids into a 293 or 293T human embryonic kidney cell line. In some aspects, DNA coding for the therapeutic gene is provided by one plasmid; the capsid proteins and replication genes issued from AAV of one or more serotypes, and helper functions issued for example from an adenovirus are all provided in trans by a second plasmid. In some aspects, DNA coding for the peptide of SEQ ID NO: 1 or a variant thereof is provided by one plasmid; the capsid proteins and replication genes issued from AAV of one or more serotypes are provided in trans by a second plasmid, and helper functions issued for example from adenovirus are provided by a third plasmid. In a particular aspect, the first plasmid comprises an expression cassette comprising a nucleic acid sequence encoding a peptide as herein described operably linked to a promoter, including two flanking inverted terminal repeats (ITRs).

Following cell culture, the gene therapy vector may be for example released from cells by freeze thaw cycles, and purified by any method well known by the skilled person, such as for example by using an iodixanol step gradient followed by ion exchange chromatography on Hi-Trap QHP columns. Then, the resulting gene therapy vector may be concentrated by spin column, and the purified vector may be stored frozen (at or below −60° C.), e.g., in phosphate buffered saline.

Related aspects of the invention include a host cell transfected or transduced with a recombinant vector, for example transduced with an AAV vector, described herein. Further related aspects of the invention include any nucleic acid molecule comprising, or consisting (essentially of), the genome of a recombinant vector, for example AAV, described herein.

### Compositions

One or more peptides herein described can be formulated for use separately or in combination, and individually or in the form of a composition. The composition may be a dietary composition or a pharmaceutical composition, and may be used in a therapeutic or prophylactic method as herein described.

In a further aspect, the present invention relates to a composition comprising a peptide, a nucleic acid, a vector or a cell herein described, and a dietarily or pharmaceutically acceptable support.

The terms “dietarily-acceptable support” relate to a carrier permitting the subject to ingest and digest without risk the composition comprising a peptide, a nucleic acid sequence, a vector or a cell as herein described, and capable of protecting said peptide from any attack, in particular related to food digestion, that could alter it before it produces its therapeutic action on the correct site and at the correct moment depending on the nature and localization of pain.

In the context of oral administration, the composition may further comprise at least one gastrointestinal protective agent, preferably an acid inhibitor, present in an amount effective to raise the gastric pH of a subject to at least 2, to at least 3, to at least 4, and more preferably to at least 5 or 6. The term “acid inhibitor” refers to agents that inhibit gastric acid secretion and increase gastric pH. The acid inhibitor may include, but is not limited to H2 blockers including cimetidine, ranitidine, ebrotidine, pabutidine, lafutidine, loxtidine, famotidine; proton pump inhibitors including omeprazole, esomeprazole, pantoprazole, lansoprazole, dexlansoprazole, rabeprazole, pariprazole, leminoprazole and tenatoprazole; or any combination thereof.

The “pharmaceutically-acceptable support/vehicle/carrier” can be a diluent, adjuvant, or excipient with which the active agent(s) (i.e. the peptide or variant thereof of the invention and optionally any additional distinct active agent) is(are) administered. Such a pharmaceutical carrier can be a sterile liquid, such as water or oil, including those of petroleum, animal, vegetable or of synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

When the pharmaceutical composition is adapted for oral administration, the tablets or capsules can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by any method well known in the art. Liquid preparations for oral administration may take the form of, for example, solutions, syrups or suspensions, or they may be presented as a dry product for reconstitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The composition of the present invention may further comprise at least one additional active compound. Preferably, the additional active compound is an active agent efficient against pain. By “efficient against pain” is meant an active agent having analgesic or antalgic properties (measurably felt by the subject). More preferably, the additional active compound is a steroidal anti-inflammatory drug (SAID), a nonsteroidal anti-inflammatory drug (NSAID) or an opioid drug.

The SAID may include, but is not limited to, hydrocortisone, cortisone, ethamethasoneb, prednisone, prednisolone, triamcinolone, dexamethasone, fludrocortisone, or any combination thereof.

The NSAID may include, but is not limited to, celecoxib, rofecoxib, lumiracoxib, valdecoxib, parecoxib, etoricoxib, CS-502, JTE-522, L-745,337, NS398, aspirin, acetaminophen (considered to be an NSAID for the purposes of the present disclosure), ibuprofen, flurbiprofen, ketoprofen, naproxen, oxaprozin, etodolac, indomethacin, ketorolac, lornoxicam, meloxicam, piroxicam, droxicam, tenoxicam, nabumetone, diclofenac, meclofenamate, mefenamic acid, diflunisal, sulindac, tolmetin, fenoprofen, suprofen, benoxaprofen, aceclofenac, tolfenamic acid, oxyphenbutazone, azapropazone, phenylbutazone, or any combination thereof.

The opioid drug may include, but is not limited to, (dextro)propoxyphene, A-methylfentanyl, alfentanil, allylprodine, bezitramide, buprenorphine, butorphanol, carfentanyl, desmethylprodine, dextromoramide, dezocine, diacetylmorphine, dihydrocodeinone, dihydroetorphine, dimorphone, diphenoxylate, dipipanone, etorphine, fentanyl, ketobemidone, lefetamine, levacetylmethadol, levomethorphan, levorphanol, loperamide, meperidine, meptazinol, methadone, methylmorphine, morphine, nalbuphine, nicomorphine, ohmefentanyl, oripavine, oxycodone, oxymorphone, PEPAP, paramorphine, pentazocine, phenazocine, piritramide, prodine, remifentanil, sufentanil, tapentadol, tilidine, tramadol, or opioid antagonists such as nalmefene, naloxone, naltrexone or any combination thereof.

### Therapeutic and Prophylactic Uses

The peptides herein described are typically used to prevent or to treat pain.

The present invention also relates to a peptide as herein described, in particular an isolated, recombinant or synthetic peptide of sequence SEQ ID NO: 1, or a peptide having at least 90% identity to SEQ ID NO: 1, for use as a medicament.

In another particular and preferred aspect, the invention relates to an isolated, synthetic or recombinant peptide of sequence SEQ ID NO: 5, or a variant thereof as herein defined, for use as a medicament.

In another aspect, inventors herein describe a peptide, nucleic acid, vector or cell as herein described for use as an active ingredient/agent for preventing or treating pain in a subject in need thereof.

In a particular aspect, inventors herein describe the use of a peptide, nucleic acid, vector, cell or composition as herein described for the manufacture of a medicament for the treatment of pain in a subject in need thereof. They also herein describe a peptide, nucleic acid, vector, cell or composition as herein described for use in the prevention or treatment of pain in a subject in need thereof, and corresponding methods for preventing or treating pain comprising a step of administering said peptide, nucleic acid, vector, cell and/or composition as herein described to a subject in need thereof.

In a particular aspect, inventors herein describe the use of a peptide, nucleic acid, vector, cell or composition as herein described for preventing or treating pain, in particular acute, sub-acute or chronic pain, preferably chronic pain.

Preferably, the pain is a neuropathic pain (such as chemotherapy-induced peripheral neuropathic pain or a chemotherapy-induced neuropathic pain), a post-operative pain, an inflammatory pain, hyperalgesia, or allodynia.

In a particular aspect, inventors herein describe the use of a peptide, nucleic acid, vector, cell or composition as herein described for preventing or treating neuropathic pain (such as chemotherapy-induced peripheral neuropathic pain or a chemotherapy-induced neuropathic pain), post-operative pain or inflammatory pain.

In a particular aspect, inventors herein describe the use of a peptide, nucleic acid, vector, cell or composition as herein described for preventing or treating chronic neuropathic pain (such as a chronic chemotherapy-induced peripheral neuropathic pain or a chronic chemotherapy-induced neuropathic pain), chronic post-operative pain or chronic inflammatory pain.

In a particular aspect, inventors herein describe the use of a peptide, nucleic acid, vector, cell or composition as herein described for preventing or treating hyperalgesia, in particular thermal (such as heat or cold hyperalgesia, preferably cold hyperalgesia) or mechanical hyperalgesia, preferably mechanical hyperalgesia even more preferably injury-induced mechanical hyperalgesia.

In another particular aspect, inventors herein describe the use of a peptide, nucleic acid, vector, cell or composition as herein described for preventing or treating allodynia, in particular mechanical allodynia. Preferably, the mechanical allodynia is a nerve injury-induced mechanical allodynia or a mechanical allodynia of the static type.

In a further particular aspect, inventors herein describe the use of a peptide, nucleic acid, vector, cell or composition as herein described for preventing or treating allodynia, in particular thermal allodynia, such as heat allodynia (i.e. perceived pain to a normally non-painful warm stimulus) or cold allodynia (i.e. perceived pain to a normally non-painful cold stimulus), preferably cold allodynia.

In a particular aspect, inventors herein describe the use of a peptide, nucleic acid, vector, cell or composition as herein described for preventing or treating mechanical hypersensitivity (also referred as hypersensitivity to mechanical stimuli or simply as hyperalgesia), preferably injury-induced mechanical hypersensitivity, in a subject in need thereof.

Treatment may result in improvements in the sensations of one or more of touch, burning or coldness, “pins and needles”, numbness, itching, excruciating pain and difficulty to correctly sense temperature. In a particular aspect, the treatment eliminates pain. In another particular aspect, the treatment reduces the symptoms of pain, in particular those of neuropathic pain (allodynia and/or hyperalgesia). The methods of the present invention render the neuropathic pain more manageable even if it does not eliminate it (i.e., improves Quality of Life).

Standard tests, well-known to the skilled person, are available in the art for assessing whether pain, in particular (chronic) neuropathic pain has been treated using a particular peptide of the invention. For example, the assessment of pain sensitivity in a subject has been standardized using quantitative sensory testing (pinpriks, pressure algometer, von Frey filaments, touch, pinching, or light pressure with the finger) or using a pain rating scale.

### Subject

In the context of the present invention, the subject or patient is an animal, preferably a mammal. In a particular aspect, the subject is a domestic animal, such as for example a horse, a dog, a cat, a cow, etc. In another particular and preferred aspect, the subject is a human-being.

The subject having chronic pain originating from a neuropathic pain may suffer from a disease classically associated with such neuropathic pain, such as for example fibromyalgia, complex regional pain syndrome, postherpetic neuralgia, Ehlers-Danlos syndrome and erythromelalgia.

In a particular aspect, the subject has fibromyalgia (FM). Fibromyalgia is a syndrome characterized by chronic musculoskeletal pain (Siracusa et al., 2021). FM is caused by a central sensitization phenomenon characterized by the dysfunction of neuro-circuits, which involves the perception, transmission and processing of afferent nociceptive stimuli, with the prevalent manifestation of pain at the level of the locomotor system. The main symptoms of this disease are muscle stiffness, joint stiffness, insomnia, fatigue, mood disorders, cognitive dysfunction, anxiety, depression, general sensitivity and the inability to carry out normal daily activities. FM can also be associated with specific diseases, such as an infection, diabetes, a rheumatic disease and/or a psychiatric or neurological disorder.

In a particular aspect, inventors herein describe the use of a peptide, nucleic acid, vector, cell or composition as herein described for preventing or treating fibromyalgia (FM).

In another aspect, the subject suffers from complex regional pain syndrome (CRPS). Complex regional pain syndrome is a chronic neurological condition involving the limbs that is characterized by severe pain along with sensory, autonomic, motor and trophic impairment (Goh et al., 2017). This condition may be induced by surgery, trauma or minor injury and has a varying course, ranging from mild and self-limiting, to chronic disease, which impairs activities of daily living and health-related quality of life. CRPS can be classified into two types: CRPS types I and II that are characterized by the absence or presence of identifiable nerve injury. CRPS type I is a syndrome that usually develops after an initiating noxious event, is not limited to the distribution of a single peripheral nerve, and is disproportionate to the inciting event. It is associated with oedema, changes in skin blood flow, abnormal sudomotor activity in the region of the pain, allodynia and hyperalgesia and commonly involves the distal aspect of the affected extremity or with a distal to proximal gradient. CRPS type II can be defined as a burning pain, allodynia and hyperpathia occurring in a region of the limb after partial injury of a nerve or one of its major branches innervating that region.

In a particular aspect, inventors herein describe the use of a peptide, nucleic acid, vector, cell or composition as herein described for preventing or treating complex regional pain syndrome (CRPS).

In still another aspect, the subject has postherpetic neuralgia (PHN). Postherpetic neuralgia is the most common complication of herpes zoster (HZ), an infection caused by the reactivation of dormant varicella zoster virus in the sensory ganglia (Ngo et al., 2020). It is characterized by a localized blistering rash and pain along the associated dermatome. PHN is defined as lingering pain for at least 90 days after the initial onset of HZ rash, and it significantly reduces the quality-of-life of affected patients. PHN is subcategorized into irritable nociceptor and deafferentation models. During the reactivation of VZV, the virus replicates and spreads from the dorsal root ganglion to its respective periphery. The propagation elicits an immune response and inflammation that damages the peripheral nerve. This damage decreases the neuron's inhibition of pain, lowering the threshold for depolarization of pain signals. This results in painful perception in response to non-painful stimuli, a process called peripheral sensitization. Repeated activation of subtype C-nociceptors also causes a heightened state of excitation in the dorsal horn. Direct viral damage by HZ weakens the descending inhibitory pain pathway, leading to a chronic activation of second-order neurons in the dorsal horn. Furthermore, loss of inhibitory gamma aminobutyric acid (GABA) producing interneurons in the dorsal horn has been reported in HZ patients with PHN when compared to HZ patients without PHN. These factors amplify all subsequent responses from the afferent input in a process called central sensitization. In PHN, this process is accompanied by the anatomical reorganization of low-threshold mechanoreceptive afferents, called Aβ-fibers, that normally relay harmless tactile stimuli to the central nervous system. When viral damage leads to the loss of C-nociceptors in the periphery, these fibers connect with second-order neurons that were originally wired to the C-nociceptor afferents in a compensatory manner. This process is called deafferentation, and patients suffering of allodynia exhibit a severe loss of sensory function.

In a particular aspect, inventors herein describe the use of a peptide, nucleic acid, vector, cell or composition as herein described for preventing or treating postherpetic neuralgia (PHN).

In still another aspect, the subject suffers from erythromelalgia (EM). Erythromelalgia is an infrequent episodic acrosyndrome affecting mainly both lower limbs bilaterally and symmetrically, or unilaterally, with the classic triad of erythema, warmth and burning pain (Maria Bibiana Leroux, 2018). EM is classified along with the chronic painful syndromes. Primary EM is an autosomal dominant inherited disorder encoded by OMIN (Online Mendelian Inheritance in Man) as #133020. It is associated with an alteration on the a subunit protein of the sodium channel type 9 (SCN9A), affecting the Nav1.7 channel that is expressed mainly in dorsal root ganglia and the sympathetic ganglia neurons. Secondary EM is associated with myeloproliferative diseases, paraneoplasias, autoimmune diseases, contact with a toxin and infections.

In a particular aspect, inventors herein describe the use of a peptide, nucleic acid, vector, cell or composition as herein described for preventing or treating erythromelalgia (EM).

In a particular aspect, inventors herein describe the use of a peptide, nucleic acid, vector, cell or composition as herein described for preventing or treating any pain-associated condition wherein the signal transduced by nociceptors or interneurons is impaired.

Inventors previously established that the absence, a reduced or insufficient expression or non-functional expression of Myosin IA (Myo1a) in a subject when compared to the expression observed in a Myo1a^{+/+} reference subject expressing a functional Myola, predisposes the subject to develop an injury-induced chronic mechanical pain and/or an inflammatory-induced chronic thermal pain (WO2017153424).

In still another aspect, the subject is a subject having one or two mutated allele in the myo1A gene. By “mutated” allele is meant a substitution, deletion or insertion in the nucleic acid of myo1A gene (in a coding or non-coding region) that alter the expression or level of expression of Myola. The mutation can affect one or more nucleobases. The substitution can a Single Nucleotide Polymorphism (SNP).

In a particular aspect, inventors herein describe the use of a peptide, nucleic acid, vector, cell or composition as herein described for preventing or treating an injury-induced chronic mechanical pain and/or an inflammatory-induced chronic thermal pain in such a subject.

### Doses

The compositions according to the invention is preferably administered directly to the subject in a therapeutically effective amount. The terms “therapeutically effective amount” refers to the amount of peptide of SEQ ID NO: 1, or of any variant thereof, required to treat, ameliorate, or prevent pain in a subject. The therapeutically effective amount can be estimated initially either in cell culture assays, or in animal models. An animal model may also be used to determine the appropriate concentration range and route of administration of the peptide. Such information can then be used to determine useful doses and routes for administration in humans.

The dosage of the peptide used in the methods herein described may vary depending on the general health condition, age, gender and weight of the subject, the nature and severity/intensity of pain, time, frequency and duration of administration, the particular peptide being used, drug combination(s), reaction sensitivities, and tolerance/response to therapy. This suitable effective dosage can be determined by routine experimentation and is under the judgement of the clinician.

To obtain suitable antalgic or analgesic effect, the effective dose of the peptide or variant thereof as herein described by inventors, per 1 kg of body weight, per 24 hours, is between 1 μg and 100 mg/kg/day, preferably between 5 μg and 80 mg/kg/day, more preferably between 10 μg and 50 mg/kg/day in an animal, typically a mammal.

When the subject is a human being, the effective dose of the peptide or variant thereof as herein described by inventors is preferably between 2.5 μg/kg/day and 0.6 mg/kg/day, preferably between 5 μg or 10 μg/kg/day and 0.5 mg/kg/day, more preferably between 50 μg or 75 μg/kg/day and 0.3 mg/kg/day in a human subject.

The dose may be administered in the bolus form or may be divided into several portions, which are administered separately along the day. In other words, the treatment can be a single dose schedule or a multiple doses schedule. The effective dose may be administered for a period of days, weeks, months or years.

Delivery of the peptide or composition as herein described to the subject may be accomplished by several routes.

In a particular aspect, the peptide or composition herein described is administered intramuscularly, intravenously, intraperitoneally, orally (per os), anally, cutaneously, subcutaneously, dermically, transdermically or intrathecally to the subject, preferably subcutaneously or orally, even more preferably orally.

As shown in the experimental part, the inventors demonstrate that the peptide of SEQ ID NO: 1, and its variant of SEQ ID NO: 5 have antalgic effect when administered subcutaneously to treat neuropathic pain, postoperative pain and/or inflammatory pain. Surprisingly, the same antalgic effect is also observed when the peptide of SEQ ID NO: 1 or 5 is administered orally (per os). Even more surprisingly and advantageously, the peptide of the invention shows better antalgic effect in comparison with the TAFA-4 (full length) protein when administered per os, which makes it more convenient and suitable for being administered to the patient than said full length protein.

Inventors herein demonstrate that a variant of SEQ ID NO:5 comprising approximately 90% of identity with the peptide of SEQ ID NO:1 achieve similar results in terms of efficacy when compared with the peptide of SEQ ID NO: 1.

### Kits

The invention also relates to a kit comprising i) a peptide, a nucleic acid, a vector, a cell and/or a composition as disclosed herein, and ii) at least one additional distinct active compound efficient against pain, preferably distinct from a peptide as herein described or variant thereof. In a particular aspect, the kit further comprises iii) written instructions for using the kit.

The kit is preferably a kit-of-parts comprising at least two parts, for example two distinct containers, wherein the first part comprises a peptide, a nucleic acid, a vector, a cell or a composition as disclosed herein and the second part comprises at least one additional distinct active compound efficient against pain. Preferably, the active compound efficient against pain is a steroidal anti-inflammatory drug (SAID), a nonsteroidal anti-inflammatory drug (NSAID) or an opioid drug as herein disclosed.

In a particular aspect, the kit comprises i) a peptide of SEQ ID NO: 1, ii) at least one additional distinct active compound efficient against pain, and iii) optionally written instructions for using the kit.

In another particular aspect, the kit comprises i) a peptide having at least 90% sequence identity to SEQ ID NO: 1 such as the peptide of SEQ ID NO: 5, ii) at least one additional distinct active compound efficient against pain, and iii) optionally written instructions for using the kit.

In one aspect, the peptide, the nucleic acid, the vector, the cell or the composition of the kit is in a form adapted for intramuscular, intravenous, intraperitoneal, oral (per os), anal, cutaneous, subcutaneous, dermical, transdermical or intrathecal routes, preferably subcutaneous or oral route, even more preferably oral route.

In another aspect, the at least one additional distinct active compound efficient against pain is in a form adapted for intramuscular, intravenous, intraperitoneal, oral (per os), anal, cutaneous, subcutaneous, dermical, transdermical or intrathecal routes.

Depending on the nature, the origin, the intensity of pain to be treated, and also depending on the nature of the peptide, nucleic acid, vector, cell or content of the composition and on the nature of the at least one additional distinct active compound efficient against pain, said products are co-administered or not, simultaneously/concomitantly or sequentially.

The present invention also relates to the use in vivo, ex vivo or in vitro of a kit of the invention for preventing or treating pain as described hereinabove, for example chronic pain, neuropathic pain, post-operative pain, inflammatory pain, hyperalgesia or allodynia. The kit of the invention may also be used in prevention or treatment of acute or subacute pain.

Also herein disclosed is the use of a kit of the invention for the manufacture of a medicament for preventing or treating pain as described hereinabove in a subject in need thereof, for example chronic pain, neuropathic pain, post-operative pain, inflammatory pain, hyperalgesia or allodynia. The kit of the invention may also be used for the manufacture of a medicament for prevention or treatment acute or subacute pain in a subject in need thereof.

### Research Tools

The present invention also includes the use of a peptide or any product herein above described as a research tool for studying pain.

The peptides herein described may be used for modulating neuronal excitability in a biological tissue or in cell cultures for example to study mechanically and/or chemically induced pain nociceptive signals.

The present invention also relates to the use of a nucleic acid encoding a peptide of SEQ ID NO: 1 or a peptide having at least 90% identity to SEQ ID NO: 1 such as the peptide of SEQ ID NO: 5, or of a vector permitting its expression, to express or modulate the (level of) expression of the peptide of SEQ ID NO: 1 or of a peptide having at least 90% identity to SEQ ID NO: 1 such as the peptide of SEQ ID NO: 5, in a biological tissue or a culture of cells.

Nucleic acid molecules herein described may also be used to create transgenic animals. This may be done locally by modification of somatic cells or through germ line therapy for the germinal cells to incorporate heritable modifications. The present invention therefore also relates to a transgenic organism (e.g. animal) comprising (i.e. containing) a nucleic acid of SEQ ID NO: 2, 6 or 9 or a variant thereof; a vector comprising a nucleic acid of SEQ ID NO:2, of SEQ ID NO: 6 or 9 or a variant thereof, or a peptide of SEQ ID NO: 1 or a peptide having at least 90% identity to SEQ ID NO: 1 such as the peptide of SEQ ID NO: 5. The invention therefore also relates to host cells or transgenic organisms containing a nucleic acid sequence encoding, or a vector permitting the expression of, any peptide as herein described.

The following examples are provided in order to demonstrate and further illustrate certain preferred aspects of the present invention and are not to be construed as limiting the scope thereof.

### EXAMPLES

### Example 1: Production by Chemical Synthesis

### Materials and Methods

### Peptides Synthesis

Native Chemical Ligation (NCL) method is used to produce the peptide of SEQ ID NO: 1. Briefly, two short peptides are produced. A first 27 amino acid N-terminal peptide having the amino acid sequence CFPGQVAGTTRAQPSCVEASIVIQKWW (SEQ ID NO: 3) is synthetized. A second 36 amino acid C-terminal peptide (containing a cysteine residue at its N-terminal part) having the amino acid sequence CHMNPCLEGEDCKVLPDYSGWSCSSGNKVKTTKVTR (SEQ ID NO: 4) is synthetized. The two peptides are ligated using a one-step ligation followed by a one-step purification.

### Mice

C57/Bl6J mice (8 to 12 weeks of age) were bought from Charles River Laboratories. Mice of both sexes were used for all experiments. As no differences were noted between males and females, the data for the two sexes were then combined. Mice were maintained under standard housing conditions (22° C., 40% humidity, 12 h light cycles, and free access to food and water). Particular efforts were made to minimize the number of mice used in this study, and the stress and suffering to which they were subjected. All experiments were conducted in line with the European guidelines for the care and use of laboratory animals (Council Directive 86/609/EEC). All experimental procedures were approved by an independent ethics committee for animal experimentation (APAFIS), as required by the French law and in accordance with the relevant institutional regulations of French legislation on animal experimentation. All experiments were performed in accordance with the ARRIVE guidelines.

### Pain Model 1: Spared Nerve Injury (SNI) Model—Neuropathic Pain.

The Spared Nerve Injury (SNI) model, developed by Decosterd and Woolf, 2000; Pain, Vol. 87, p 149-158 was used. It was used as a neuropathic pain model. The SNI model consists in the transection of tibial branches and of the common peroneal nerve of the sciatic nerve: the sural nerve remaining intact. The latter then develops signs of neuropathic pains with substantial mechanical allodynia. The SNI model has many advantages:
Neuropathic pain is persistent. This allows to grasp habituation phenomena upon repeated injections of the peptide.
The generated pain is robust.
The model is very reproducible.

Mice were anesthetized with ketamine (100 mg/kg IP) and xylazine (10 mg/kg IP) and the left sciatic nerve was exposed under aseptic conditions. The distal trifurcation of the sciatic nerve was identified and the tibial and common peroneal branches were ligated with polypropylene nonabsorbable 6-0 sutures (Ethicon); 1 mm was cut out, leaving the sural branch intact. The wound was closed with sutures, and the animals were allowed to recover and returned to their cages.

### Pain Model 2: Paw Incision—Postoperative Pain.

Paw incision surgery was performed as described by Brennan and co-workers (1999) (Brennan, 1999). Mice were anesthetized with ketamine (100 mg/kg IP) and xylazine (10 mg/kg IP) and a longitudinal incision was made through the skin and fascia of the right hind paw. Forceps were used to elevate the flexor digitorum brevis muscle longitudinally and an incision was made through the muscle with a scalpel, to cut it into two halves. The wound was closed with sutures, and the animals were allowed to recover and returned to their cages. The paw incision was used as postoperative pain model.

### Pain Model 3: Carrageenan Injection—Inflammatory Pain.

Inventors injected 20 μl of 1% λ-carrageenan (Sigma-Aldrich, 22049-5G-F) in 1×PBS into the plantar surface of the left hind paw of the mouse with a Hamilton syringe. The carrageenan injection was used as an inflammatory pain model.

### Von Frey's Test

The Von Frey's uses Von Frey (VF) hair or fibers, which are small pieces of nylon rod, approximately 50 mm in length to test a rodent's sensitivity to a mechanical stimulus. In this test, the animal stands on an elevated mesh platform, and the Von Frey hairs are inserted through the mesh to poke the animal's hindpaw. Normal reactions for the animal include withdrawing or licking or shaking the paw. The “up-down” Von Frey method is used to determine the mechanical force required to elicit a paw withdrawal response in 50% of animals. Here, mice were placed in plastic chambers on a wire mesh grid and stimulated with von Frey filaments (Bioseb) by the “up-down” method (45) starting with a 1 g filament, and using 0.04 and 4 g filaments as the cutoffs.

### Statistical Analysis

The results are expressed as means±SEM. Statistical analyses were performed with Prism 7 (Graphpad Software, La Jolla, CA, USA).

### Results

### Pain Model 1: Spared Nerve Injury (SNI) Model—Neuropathic Pain.

### 1.1 Antalgic Effect of Peptide of SEQ ID NO:1 Administered Subcutaneously

The purpose of these experiments was to assess the antalgic effect of the peptide of SEQ ID NO: 1 according to the invention (herein identified as “TT1”) by subcutaneous injection in the Spared Nerve Injury (SNI) model, which generates neuropathic pain.

The experiments were conducted on eight-week-old male WT C57Bl6 mice. Three groups of 8 mice were used. Peptide of SEQ ID NO: 1 is resuspended in 0.9% NaCl at [0.6 mg/mL]. The peptide was diluted and used at a concentration of 0.3 mg/kg in this study.

0.9% NaCl solution is used as a negative control (vehicle) and 5 mg/kg of pregabalin, a well-known drug used to treat neuropathic pain, is used as a positive control.

After having measured the base threshold of mice with Von Frey (VF) filaments by the up/down method, the SNI model is set into place. The mice are anesthetized, ligature of the tibial nerve and of the fibular nerve is put into practice and these two nerves are then severed. The sural nerve left intact develops neuropathy quite rapidly. The occurrence of neuropathy is ascertained after 3 days post-surgery. A decrease in the response threshold to Von Frey filaments of the ipsilateral paw is thereby observed.

7 days after surgery, the response threshold is again measured. 100 μl/10 g of each of the peptide solutions, of vehicle and pregabalin solution are then blind-injected subcutaneously for the experimenter.

The response threshold is measured after 1 hour, 2 hours, 4 hours, and then 24 h after injection.

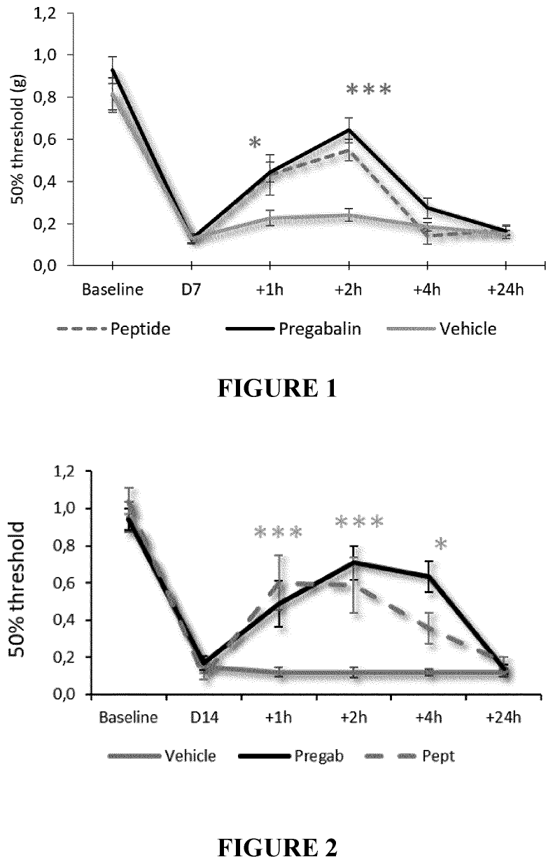

At day 7 post-SNI, all mice exhibit a drastic decrease in the mechanical thresholds (FIG. 1). The subcutaneous injection of the peptide of SEQ ID NO: 1 (referred as “Peptide” or “TT1”) induced a strong increase in the response (i.e., paw withdrawal) threshold as soon as 1 hour post-administration with a maximum increase at 2 hours with values comparable to pregabalin. Statistical analysis shows strong significant result (with p value <0.001) at 2 hours in comparison with negative control.

### 1.2 Antalgic Effect of Peptide of SEQ ID NO: 1 Administered Per Os

The same protocol was used as described in 1.1 except that the administration of the peptide, vehicle and pregabalin was done 14 days after surgery using per os administration for the peptide and vehicle.

For per os administration, the peptide of SEQ ID NO: 1 was diluted at a concentration of 30 μg/mL in a solution of 1% w/v Hydroxypropylmethylcellulose (Sigma-aldrich #423238, batch MKCD3665), 0.5% v/v Tween 80 (Euromedex #2002-A, batch 100412/16S407), referred then after as HPMC solution (vehicle).

Vehicle and peptide solution were orally administered using two gavage needles. Pregabalin (5 mg/kg) was subcutaneously injected, by a different experimenter, so the experimenter performing the VF measures was blind for the treatment (n=8 for each treatment). Administrations were performed at 14 days after surgery.

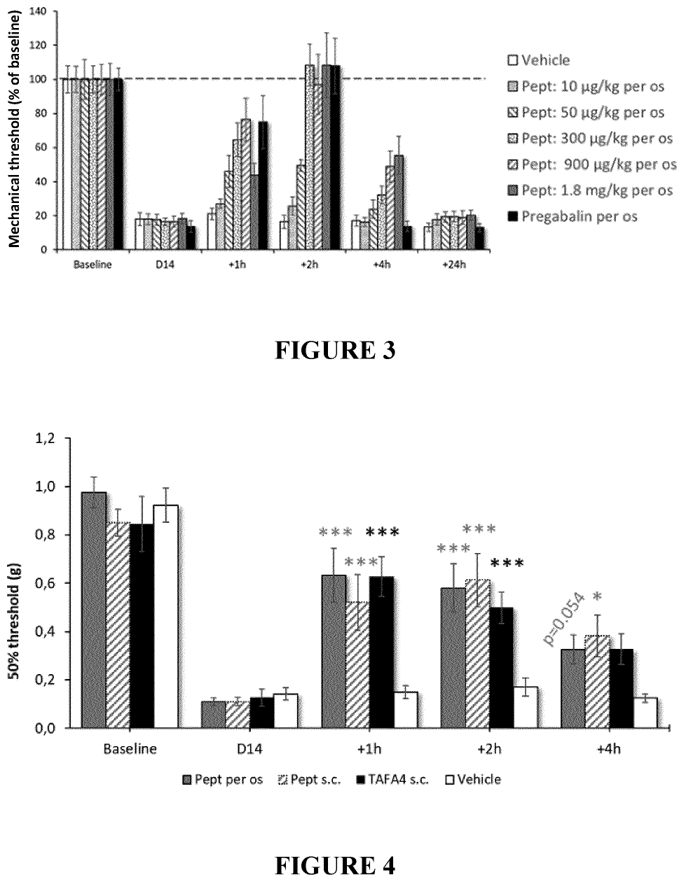

At day 14 post-SNI, all mice exhibit a drastic decrease in the mechanical thresholds (FIG. 2).Oral administration of the vehicle had no effect. The per os administration of peptide of SEQ ID NO: 1 also induced a strong increase in the response (i.e., paw withdrawal) threshold as soon as 1 hour post-administration with a slow decrease until 24 hours. It was significantly different compared to vehicle-treated mice up to 4 h (with p value <0.001 at 1 and 2 hours and p value <0.05 at 4 hours).

### 1.3 Dose-Dependent Antalgic Effect of Peptide of SEQ ID NO: 1 Administered Per Os

The peptide of SEQ ID NO: 1 is resuspended in HMPC solution at 5 different concentrations (1, 5, 30, 90 and 180 μg/mL).

As previously described, the Von Frey filament measurement with the up/down method is done for determining the baseline. Then, the SNI model is set into place. The response threshold fourteen days (DAY 14—D14) after surgery is measured to check for the occurrence of neuropathic pain. Then, blind per os administration of 100 μl/10 g of peptide solution at 5 different concentrations (n=8 for 10 μg/kg; n=8 for 50 μg/kg; n=13 for 300 μg/kg n=8 for 900 μg/kg, n=8 for 1.8 mg/kg) is performed. The response threshold is measured after 1 hour, 2 hours, 4 hours, and then 24 h after oral administration. Pregabalin (5 mg/kg, n=6) and vehicle (n=9) were also administered by gavage.

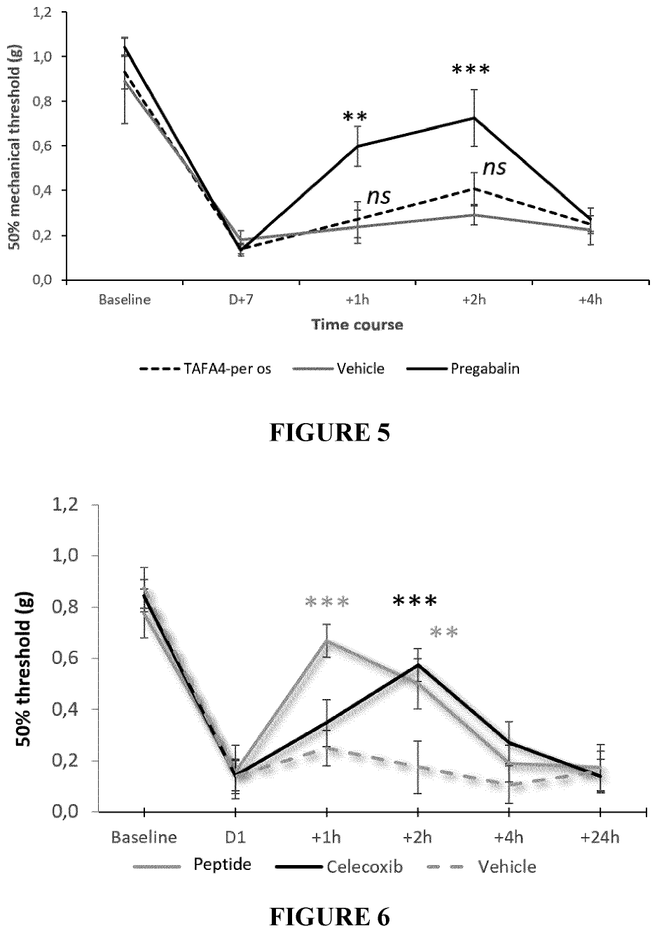

At day 14 post-SNI, all mice exhibit a drastic decrease in the mechanical thresholds, illustrative of a strong mechanical hypersensitivity, as compared to the baseline thresholds (FIG. 3). Oral administration of the vehicle had no effect. The antalgic effect of peptide of SEQ ID NO: 1 is dose-dependent, with the maximum reversal effect on mechanical threshold observed at 2 h with the 300 μg/kg dose. Interestingly, the two highest doses (0.9 and 1.8 mg/kg) produced the same effect at 2 h but also lasted for a longer time with still a significant effect at 4 h post-administration. The maximum reversal effect of pregabalin on mechanical threshold is observed at 2 h post-administration; this effect is completely abolished at 4 h post-administration.

1.4 Comparison of the Antalgic Effect of TAFA-4 (Full Length Protein) with Peptide of SEQ ID NO: 1 Administered Subcutaneously or Per Os

The inventors have compared the antalgic effect of the peptide of the invention with the TAFA-4 (full length) protein of the prior art. The same protocol was used as described in 1.1 and 1.2 except that the administration of the peptide and of the TAFA-4 (full length) protein was done 14 days after surgery.

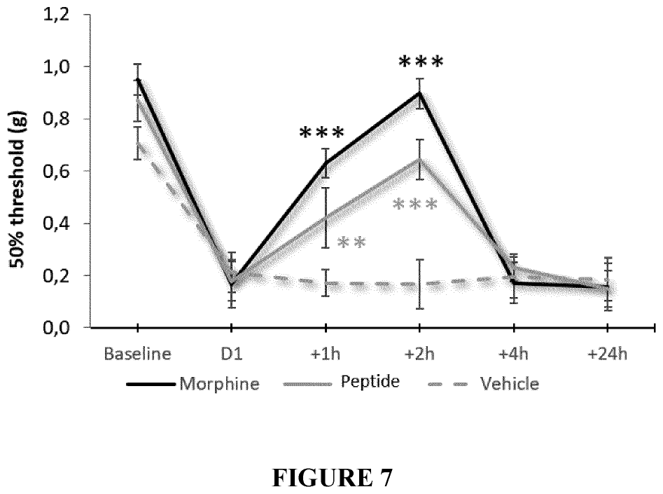

The peptide of the invention surprisingly maintains the same antalgic effect as the TAFA-4 full length protein when administered subcutaneously (FIG. 4).

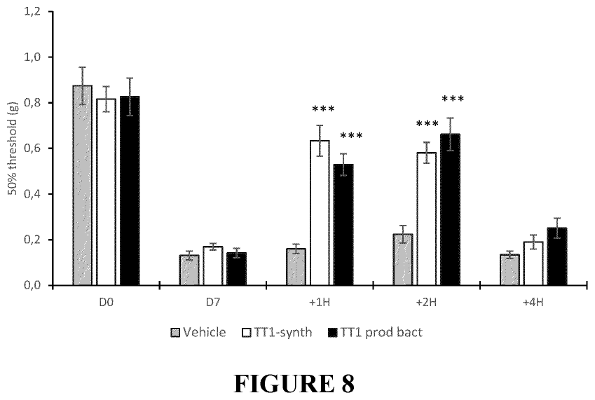

The inventors made also the comparison between the TAFA-4 full length protein and the peptide of the invention when administered per os. The same experiment was conducted with the TAFA-4 full length protein (the administration of the TAFA-4 protein was done 7 days after surgery). The TAFA-4 full length protein did not show significant antalgic effect when administered orally (i.e., per os) (FIG. 5).

From the results appearing on FIGS. 4 and 5 it can be concluded that, when administered per os, the peptide of the invention has a much better antalgic effect than the TAFA-4 (full length) protein.

Altogether, example 1 shows that the peptide of the invention induced an antalgic effect by subcutaneous and per os administration in the SNI model (neuropathic pain model). Advantageously the peptide of the invention maintains its activity as compared to the TAFA-4 (full length) protein when administered subcutaneously. Even more surprisingly, the peptide of the invention shows better antalgic effect than the TAFA-4 (full length) protein when administered per os.

### Pain Model 2: Carrageenan Injection—Inflammatory Pain.

The experiments were conducted on eight-week-old male WT C57Bl6. Three groups of 8 mice were used. Peptide of SEQ ID NO: 1 is resuspended in HMPC solution at concentrations of 30 μg/mL, for a 10 μl injection per gram.

As previously described, measurement with Von Frey filaments by the up/down method for determining the baseline was done. Then, an intraplantar injection of 20 μl of carrageenan (1%) was performed into a hind paw. The response threshold, 24 h after injection (D1), was measured followed by per os administration of the peptide solution at 0.3 mg/kg (n=8), or vehicle solution (n=8) or subcutaneous injection of celecoxib solution at 20 mg/kg (n=8), blind for the experimenter. Celecoxib is a well-known drug used as positive control. It is a COX-2 inhibitor and nonsteroidal anti-inflammatory drug (NSAID) used to treat the pain and inflammation in several diseases. The response threshold was measured 1 h, 2 h, 4 h and 24 h after administration of the peptide or celecoxib.

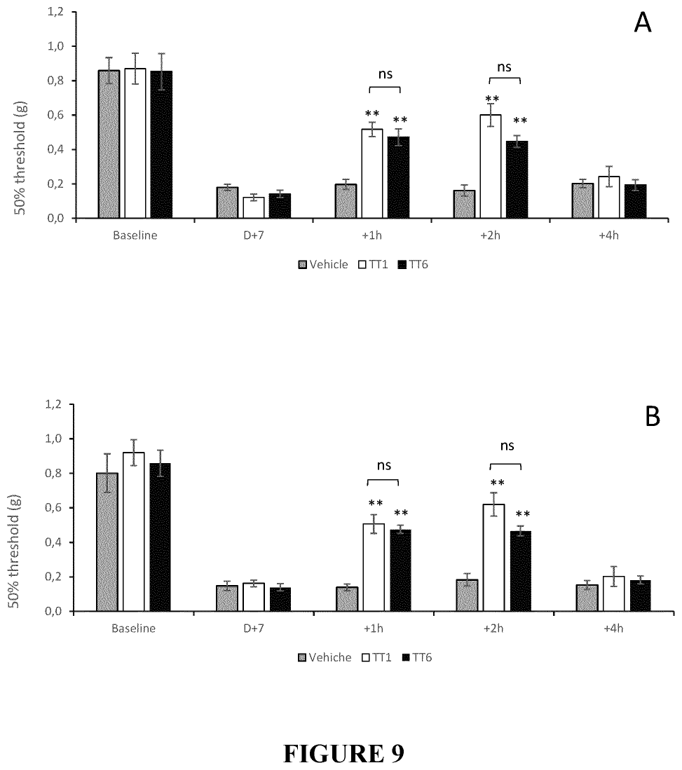

After injection of carrageenan, the mice developed mechanical allodynia (see at D1) (FIG. 6). Subcutaneous administration of positive control (celecoxib) causes an increase in the response threshold. Similarly, oral (per os) administration of the peptide of the invention also induced a statistically significant increase in the response threshold. Interestingly the maximum antalgic effect of the peptide is obtained after 1 h whereas it takes 2 hours with regards to celecoxib.

These results show that the peptide of the invention caused an antalgic effect via per os administration in the carrageenan model (inflammatory pain model). Advantageously, the peptide of the invention had a rapid onset of the antalgic effect.

### Pain Model 3: Paw Incision—Postoperative Pain.

The experiments were conducted on eight-week-old male WT C57Bl6. Three groups of 8 mice were used. Peptide of SEQ ID NO: 1 is resuspended in HMPC solution NaCl at concentrations of 30 μg/mL, for a 10 μl injection per gram.

As previously done, measurement with Von Frey filaments by the up/down method for determining the baseline was done. Then, paw incision surgery was performed according to the protocol described above. The response threshold, 24 h after injection (D1), was measured followed by per os administration of the peptide solution at 0.3 mg/kg (n=8), vehicle solution (n=8) or morphine solution (positive control) at 1 mg/kg (n=8), blind for the experimenter. The response threshold was measured 1 h, 2 h, 4 h and 24 h after administration.

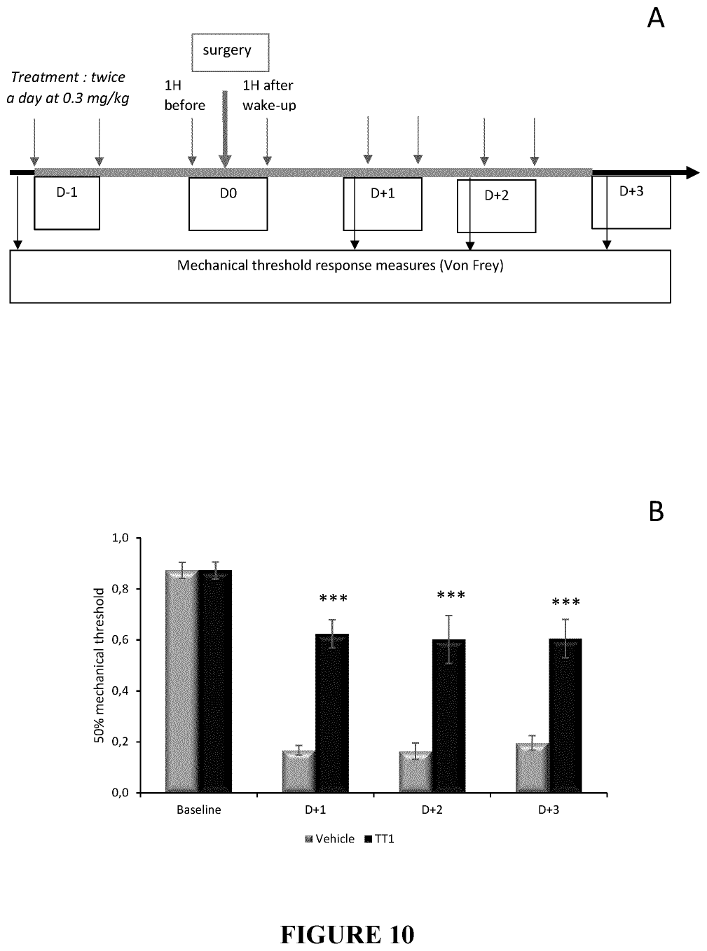

After paw incision, the mice developed mechanical allodynia (see at D1 on FIG. 7). Oral administration of positive control (morphine) causes an increase in the response threshold. Similarly, oral administration of the peptide of the invention also induced a statistically significant increase in the response threshold 1 hour post-administration, with a maximum effect occurring at 2 hours post-administration.

These results show that the peptide of the invention caused an antalgic effect by per os administration in the paw incision model (postoperative pain model).

### Conclusion:

Altogether Inventor's results show the antalgic effect of the peptide of SEQ ID NO: 1 in a neuropathic, inflammatory and post-operative pain model. Mechanical allodynia (decrease in the response threshold) induced by these models may be inhibited by subcutaneous injection or oral (per os) administration of said peptide. Inventors also demonstrated that the antalgic effect of peptide of SEQ ID NO: 1 is dose-dependent.

Advantageously, the peptide of the invention, while being much easy (and consequently cheaper) to produce and to obtain than the full length TAFA-4 protein, maintains its antalgic activity in comparison with the latter when administered subcutaneously. Even more surprisingly and advantageously, when it comes to oral (per os) administration, the peptide of the invention shows better antalgic effect in comparison with the TAFA-4 (full length) protein which makes it more convenient and suitable for being administered to the patient than the TAFA-4 full length protein.

### Example 2: Bioproduction of TT1 and its Variant TT6

The inventors produced the peptides of SEQ ID NO: 1 (“TT1”) and SEQ ID NO: 5 (“TT6”) by the recombinant route (“TT1 bioproduct” and “TT6 bio-product”) using the protocol described herein below.

The amino acid sequence of the peptide of SEQ ID NO: 5 is 90.48% identical to the amino acid sequence of the peptide of SEQ ID NO: 1.

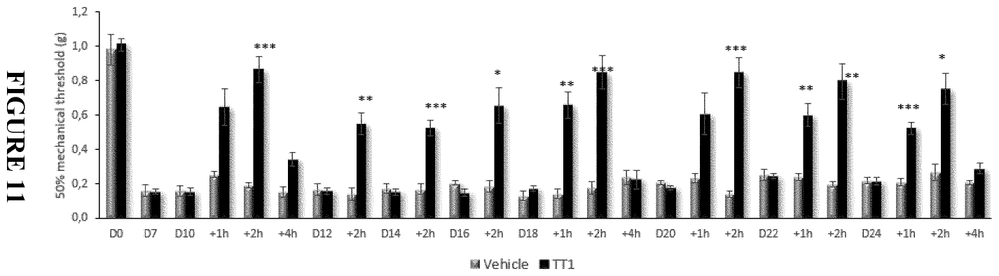

Using the behavioral assay described in example 1 (pain model 1: SNI), the inventors obtained confirmation that the synthetically obtained TT1 and bioproduced TT1 peptides thus obtained have the same pain-relieving efficacy (see FIG. 8). They also obtained confirmation that both the bioproduced TT1 and TT6 peptides thus obtained have the same pain-relieving efficacy as that observed with the TT1 peptide obtained synthetically (see FIG. 9).

### Materials

Plasmid pEt28A+, kanamycin-resistant
E. coli BL21 (DE3) pLysS strain, competent, chloramphenicol-resistant
Recombinant 6×His-TEVprotease, produced and purified in the AFMB lab (academic MTA)
NZY Auto-induction LB medium (powder): nzytech (MB17903)
2×YT Broth (powder): MP Biomedicals (3012032)
Sonicator, centrifuge, Petri dishes (d 10 cm)
MF-Millipore membrane, cellulose ester, hydrophilic, 0.22 μm, 47 mm, white: MERCK
HisTrap excel 5 mL cartridge: Cytiva
Imidazole buffer substance ACS. CAS 288-32-4: Millipore (MERCK)
Dialysis tubing Zellu/Trans/ROTH T2: MWO 6000-8000, 50 mm, 30 m: CARL ROTH
Affinity chromatography media, Chelating Sepharose™ Fast Flow: Cytiva
AMICON Ultra-15 Centrifugal Filter Unit, 3K: Millipore
Superdex S200 increase 10/300 GL column: Cytiva
SDS-PAGE gel with 20% reticulation

### Methods

### 1. Production of the 6×His-(TEV Site)-TT1 (or 6×His-(TEV Site)-TT6) Fusion Protein (Herein Respectively Identified as SEQ ID NO: 7 and 8)

a. Transformation of E. coli BL21 (DE3) pLysS Strain
Thaw competent bacteria BL21 (DE3) pLySs, resistant to chloramphenicol
Add 1 μL of plasmid (1000 ng/μL) per 50 μL of competent bacteria
Keep 5 min on ice
Perform thermal shock: 1 min at 37° C.
Keep 5 min on ice
Add 500 μL of NZY Auto-induction LB medium or 2YT Broth media (in sterile condition)
Incubate at 37° C. for at least 30 min with stirring (200 rpm)
Heat LB agar until complete dissolution
   For 2 Petri dishes, mix and poor:
      40 mL of melted LB agar
      40 μL of kanamycin, for the plasmid
      40 μL of chloramphenicol, for the strain
   Once solidified, spread the transformed bacteria, 50-100 μL per dish
      b. Preculture

For 500 mL of culture:
30 mL of NZY Auto-induction LB medium or 2YT Broth media
30 μL of kanamycin, for the plasmid
30 μL of chloramphenicol, for the strain
1 colony

Incubate overnight at 37° C. with stirring (200 rpm)

c. Culture
In a 2 L Erlenmeyer flask:
With NZY Auto-induction LB medium
   500 mL of media
   500 μL of kanamycin, for the plasmid
   500 μL of chloramphenicol, for the strain
   A volume of preculture to reach an OD600 nm of ca. 0.1
   Incubate at 37° C. with stirring (200 rpm) until OD600 nm reaches ca. 0.6-0.8 Incubate overnight at 17° C. with stirring (200 rpm).
With 2YT Broth media
   500 mL of media
   500 μL of kanamycin, for the plasmid
   500 μL of chloramphenicol, for the strain
   A volume of preculture to reach an OD600 nm of ca. 0.1
   Incubate at 37° C. with stirring (200 rpm) until OD600 nm reaches ca. 0.6-0.8.
   Add 500 μL of IPTG (stock solution 1 M)
   Incubate overnight at 17° C. with stirring (200 rpm)
Centrifuge at 5000 rpm for 20 min at room temperature
Resuspend the pellets in 50 mL of lysis buffer: Tris 50 mM pH 8.0, NaCl 300 mM, imidazole 10 mM pH 8.0, lysozyme 0.5 mg/mL
Flash-freeze in liquid nitrogen
Store at −80° C.

### 2. Purification and Cleavage of the Fusion Protein

a. Cell Lysis: Total Lysis
Thaw a stored pellet (resuspended in lysis buffer) for 10 min at 37° C.
Add DNAse 1/100 (v/v) (2 mg mL stock-solution) and MgSO₄ 1/200 (v/v) (2M stock solution)
Shake/incubate for 20 minutes at 4° C.
Sonicate for 6 min, such as 30 sec ON, 30 sec OFF, 40% amplitude (the beaker containing the sample should be kept on ice)
Centrifuge at 20 000 rpm for 40 min at 4° C.
Filter the supernatant through a 0.22 μm filter membrane
   b. Standard Immobilized Metal Chelation Chromatography (IMAC) (AKTA Purifier)
Equilibrate a HisTrap excel 5 mL column with buffer A: Tris 50 mM pH 8, NaCl 300 mM, imidazole 10 mM pH 8) with a flow rate of 3 mL/min
Load the supernatant, and then wash the column with 20 CV i.e. 100 mL of buffer A
Elute the fusion protein with 10 CV i.e. 50 mL of buffer B: Tris 50 mM pH 8, NaCl 300 mM, imidazole 250 mM pH 8)
Measure the volume and OD280 nm of the pooled elution fractions—calculate concentration and amount using A1‰=1.1 UDO/mg at 280 nm (if no disulphide bonds)
Using a dialysis tubing with a 6-8 kDa cut-off: dialyze the elution pool overnight at 4° C. against 2 L of buffer A: Tris 50 mM pH 8, NaCl 1 M, imidazole 10 mM pH 8)
   c. Cleavage
Measure the OD280 nm of the dialyzed elution
For the cleavage to take place correctly, the elution must be diluted to have a concentration between 0.5-0.7 mg/mL
Add recombinant TEV (1/10 w/w) in elution
Incubate for 4 hr at room temperature
Incubate overnight at 4° C.
Add recombinant TEV (1/20 w/w) in elution
Incubate for 4 hr at room temperature
Incubate overnight at 4° C.
Centrifuge for 15 min at 4° C. at 7500 rpm to remove any precipitate

### 3. Purification of TT1 or TT6

a. Resin Preparation

For a sample issued from a 1L bacteria culture:
4 mL of resin
800 μL of NiSO₄ 0.2 M and incubate for 15-30 min
Wash with 15 mL of H₂O and centrifuge for 5 min at 5 000 rpm at 4° C.
Discard the supernatant
Wash with 15 mL of H₂O and centrifuge for 5 min at 5 000 rpm at 4° C.
Discard the supernatant
Wash with 15 mL of Tris 50 mM pH 8.0, NaCl 1 M and centrifuge for 5 min at 5 000 rpm at 4° C.
Discard the supernatant
Repeat the operation until the liquid is transparent
Wash with 15 mL of Tris 50 mM pH 8.0, NaCl 1 M, imidazole 10 mM pH 8.0, incubate for 10 min and centrifuge for 5 min at 5 000 rpm at 4° C.
Discard the supernatant
Wash with 15 mL of Tris 50 mM pH 8.0, NaCl 1 M, imidazole 10 mM pH 8.0, incubate for 10 min and centrifuge for 5 min at 5 000 rpm at 4° C.
Discard the supernatant
Add 15 mL of Tris 50 mM pH 8.0, NaCl 1 M, imidazole 10 mM pH 8.0, incubate overnight at 4° C.

### b. “Flow-Through” Immobilized Metal (Ni²⁺) Chelation Chromatography (IMAC) (Batch)

Incubate the cleaved sample with the pre-equilibrated resin for 2 hr at 4° C.
Transfer the resin/cleaved elution mixture to an empty column
Collect the flow-through, that contains TT1, or TT6, only (the residual fusion protein—if any—and the TEV remain on the column)
   c. Concentration
Concentrate the flow-through using an AMICON 3K concentrator (3900 rpm, 4° C.) until a volume of 200-500 μL is obtained (the smaller the better)
Centrifuge at 10 000 rpm for 5 min to get rid of any precipitate, transfer supernatant to a clean tube
   d. Size-Exclusion Chromatography (SEC) (AKTA Purifier)
Equilibrate a Superdex S200 increase 10/300 GL column with buffer Tris 50 mM pH 8.0, NaCl 1 M, imidazole 10 mM pH 8.0: using a flow rate of 0.5 mL/min
Load the concentrated flow-through and elute (TT1 and TT6 begin to elute at ca. 19 mL)
Measure the volume and OD280 nm of the pooled TT1, or TT6, fractions—calculate concentration and amount using A1‰=2.6 UDO/mg at 280 nm (if no disulphide bonds)
   e. SDS-PAGE (20% Reticulation Gel, Reducing Conditions)
For each purification step and each elution peak: take off 1-4 μg protein, denature (SDS) and reduce (DTT or 2/beta-mercaptoethanol) samples and store at 4° C. or −20° C. until final use
Load on SDS-PAGE gel and migrate (200 V)

### Example 3: Preventive Antalgic Effect of TT1

The purpose of this experiment was to assess whether the peptide of SEQ ID NO: 1 (“TT1”) has also a preventive antalgic effect by subcutaneous injection of TT1 in the paw incision model (as described above).

### Materials and Methods

TT1 or vehicle were subcutaneously administered twice a day at different time point: the day before surgery (D-1), 1 hour before and 1 hour after surgery (and wake-up) at D day, and at D+1 and D+2 (FIG. 10A). Mechanical threshold response measurements at D+1 (before TT1 injection at D+1), D+2 (before TT1 injection at D+2) and D+3 were performed.

### Results

After paw incision, mice treated with vehicle developed mechanical allodynia (see at D1, D2 and D3 on FIG. 10B). On the contrary, mice treated with TT1 did not develop mechanical allodynia. In comparison with mice treated with vehicle, mice treated with TT1 presents a statistically significant higher response threshold at D1, D2 and D3.

These results show that the peptide TT1 of the invention can be used preventively in a paw incision model (postoperative pain model).

### Example 4: Absence of Tolerance after Per Os Repeated Administration of TT1 on CCI Neuropathic Pain Model

The purpose of this experiment was to assess whether the subject treated with the peptide of SEQ ID NO: 1 (“TT1”) may induce a tolerance towards this peptide.

### Materials and Methods

CCI neuropathic pain model: Chronic Constriction Injury (CCI) was performed as described previously by Bennett and Xie 1988 (A peripheral mononeuropathy in rats that produces disorders of pain sensation like those in man. Pain vol 33: pp 87-107). Briefly, unilateral peripheral mononeuropathy was induced in mice anaesthetized with Ketamine/Xylasine (respectively 100 mg/kg and 10 mg/kg ip) with two ligatures (6-0 Monocryl, Ethicon) tied loosely (with about 1 mm spacing) around the common sciatic nerve. The nerve was constricted to a barely discernible degree, so that circulation through the epineural vasculature was not interrupted.

After establishment of CCI neuropathic pain model as described above, mice were daily treated with the peptide of SEQ ID NO:1 (“TT1”) or vehicle for 14 consecutive days starting 10 days after surgery (D10). Antalgic effect of TT1 was determined every two days until the 24^{th} day after surgery (D24).

### Results

The subcutaneous injection of the peptide of TT1 induced a strong increase in the response (i.e., paw withdrawal) threshold demonstrating the antalgic effect of TT1. Interestingly, the antalgic effect of TT1 remains constant for the entire duration of the treatment (14 days) without any decrease in TT1 efficacy, even at D24. These results show that the peptide TT1 of the invention can be used repeatedly over a long period of time without inducing a tolerance in a subject.

### REFERENCES

Benyamin, R., Trescot, A. M., Datta, S., Buenaventura, R., Adlaka, R., Sehgal, N., Glaser, S. E., and Vallejo, R. (2008). Opioid complications and side effects. Pain Physician 11, S105-120.
Bourane, S., Duan, B., Koch, S. C., Dalet, A., Britz, O., Garcia-Campmany, L., Kim, E., Cheng, L., Ghosh, A., Ma, Q., et al. (2015a). Gate control of mechanical itch by a subpopulation of spinal cord interneurons. Science 350, 550-554.
Bourane, S., Grossmann, K. S., Britz, O., Dalet, A., Del Barrio, M. G., Stam, F. J., Garcia-Campmany, L., Koch, S., and Goulding, M. (2015b). Identification of a spinal circuit for light touch and fine motor control. Cell 160, 503-515.
Boyle, K. A., Gradwell, M. A., Yasaka, T., Dickie, A. C., Polgar, E., Ganley, R. P., Orr, D. P. H., Watanabe, M., Abraira, V. E., Kuehn, E. D., et al. (2019). Defining a Spinal Microcircuit that Gates Myelinated Afferent Input: Implications for Tactile Allodynia. Cell reports 28, 526-540 e526.
Colloca, L., Ludman, T., Bouhassira, D., Baron, R., Dickenson, A. H., Yarnitsky, D., Freeman, R., Truini, A., Attal, N., Finnerup, N. B., et al. (2017). Neuropathic pain. Nat Rev Dis Primers 3, 17002.
Costigan, M., Scholz, J., and Woolf, C. J. (2009). Neuropathic pain: a maladaptive response of the nervous system to damage. Annu Rev Neurosci 32, 1-32.
Coull, J. A., Beggs, S., Boudreau, D., Boivin, D., Tsuda, M., Inoue, K., Gravel, C., Salter, M. W., and De Koninck, Y. (2005). BDNF from microglia causes the shift in neuronal anion gradient underlying neuropathic pain. Nature 438, 1017-1021.
Delfini, M. C., Mantilleri, A., Gaillard, S., Hao, J., Reynders, A., Malapert, P., Alonso, S., Francois, A., Barrere, C., Seal, R., et al. (2013). TAFA4, a chemokine-like protein, modulates injury-induced mechanical and chemical pain hypersensitivity in mice. Cell reports 5, 378-388.
Duan, B., Cheng, L., Bourane, S., Britz, O., Padilla, C., Garcia-Campmany, L., Krashes, M., Knowlton, W., Velasquez, T., Ren, X., et al. (2014). Identification of spinal circuits transmitting and gating mechanical pain. Cell 159, 1417-1432.
Goh, E L., Chidambaram, S., Ma, D. (2017). Complex regional pain syndrome: a recent update. Burns & Trauma 5:2.
Leroux, Maria Bibiana (2018). Erythromelalgia: a cutaneous manifestation of neuropathy? An Bras Dermatol. 93(1):86-94.
Ngo, A L., Urits, I., Yilmaz, M., Fortier, L., Anya, A., Oh, JH., Berger, AA., Kassem, H., Sanchez, MG., Kaye, A D, et al. (2020). Postherpetic Neuralgia: Current Evidence on the Topical Film-Forming Spray with Bupivacaine Hydrochloride and a Review of Available Treatment Strategies. Adv Ther. 37(5): 2003-2016.
Peirs, C., Williams, S. P., Zhao, X., Walsh, C. E., Gedeon, J. Y., Cagle, N. E., Goldring, A. C., Hioki, H., Liu, Z., Marell, P. S., et al. (2015). Dorsal Horn Circuits for Persistent Mechanical Pain. Neuron 87, 797-812.
Petitjean, H., F, B. B., Tsao, D., Davidova, A., Sotocinal, S. G., Mogil, J. S., Kania, A., and Sharif-Naeini, R. (2019). Recruitment of Spinoparabrachial Neurons by Dorsal Horn Calretinin Neurons. Cell reports 28, 1429-1438 e1424.
Petitjean, H., Pawlowski, S. A., Frame, S. L., Sharif, B., Hamad, D., Fatima, T., Berg, J., Brown, C. M., Jan, L. Y., Ribeiro-da-Silva, A., et al. (2015). Dorsal Horn Parvalbumin Neurons Are Gate-Keepers of Touch-Evoked Pain after Nerve Injury. Cell reports 13, 1246-1257.
Sarver, D. C., Lei, X., and Wong, G. W. (2021). FAM19A (TAFA): An Emerging Family of Neurokines with Diverse Functions in the Central and Peripheral Nervous System. ACS Chem Neurosci 12, 945-958.
Siracusa, R., Di Paola, R., Cuzzocrea, S., Impellizzeri, D. (2021). Fibromyalgia: Pathogenesis, Mechanisms, Diagnosis and Treatment Options Update. Int J Mol Sci. 22(8): 3891.
Tom Tang, Y., Emtage, P., Funk, W. D., Hu, T., Arterburn, M., Park, E. E., and Rupp, F. (2004). TAFA: a novel secreted family with conserved cysteine residues and restricted expression in the brain. Genomics 83, 727-734.
Zeilhofer, H. U., Wildner, H., and Yevenes, G. E. (2012). Fast synaptic inhibition in spinal sensory processing and pain control. Physiol Rev 92, 193-235.
Zhang, Y., Liu, S., Zhang, Y. Q., Goulding, M., Wang, Y. Q., and Ma, Q. (2018). Timing Mechanisms Underlying Gate Control by Feedforward Inhibition. Neuron 99, 941-955 e944.

1-17. (canceled)

18. An isolated, synthetic or recombinant peptide of sequence SEQ ID NO: 1, or a peptide having at least 90% identity to SEQ ID NO: 1.

19. The peptide of claim 18, wherein the peptide having at least 90% identity to SEQ ID NO: 1 modulates excitability of spinal cord interneurons.

20. The peptide of claim 19, wherein amino acids Q and Y appearing in bold in sequence CFPGQVAGTTRAQPSCVEASIVIQKWWCHAMNPCLEGEDCKVLPDYSGWSC SSGNKVKTTKVTR (SEQ ID NO: 1) remain unchanged in the amino acid sequence of the peptide having at least 90% identity to SEQ ID NO: 1.

21. The peptide of claim 18, wherein the peptide is a recombinant peptide.

22. The peptide of claim 18, wherein the peptide is a peptide of SEQ ID NO: 5.

23. A nucleic acid sequence encoding the peptide of claim 18.

24. A vector comprising the nucleic acid sequence of claim 23.

25. A cell comprising a nucleic acid sequence according to claim 23 or a vector comprising said nucleic acid sequence.

26. A composition comprising the peptide of claim 18, a nucleic acid encoding said peptide, a vector comprising said nucleic acid or a cell comprising said nucleic acid and a dietarily or pharmaceutically acceptable support.

27. The composition of claim 26, further comprising at least one additional active compound.

28. The composition of claim 27, wherein said one additional active compound is a SAID, NSAID or opioid drug.

29. A method of treating pain in a subject comprising the administration of the peptide of claim 18 or a composition comprising at least one additional active compound and said peptide, a nucleic acid encoding said peptide, a vector comprising said nucleic acid or a cell comprising said nucleic acid and a dietarily or pharmaceutically acceptable support to a subject in need of treatment.

30. The method of claim 29, wherein said pain is a chronic pain, a neuropathic pain, a postoperative pain, an inflammatory pain, hyperalgesia, or allodynia.

31. The method of claim 29, wherein the subject is a mammal or a human being.

32. The method of claim 29, wherein the subject has at least one mutated allele in the myo1A gene.

33. The method of claim 29, wherein the dose of peptide is between 1 μg and 100 mg/kg/day in a mammal or is between 2.5 μg/kg/day and 0.6 mg/kg/day in a human subject.

34. The method of claim 29, wherein said peptide or composition is administered intramuscularly, intravenously, intraperitoneally, orally (per os), anally, cutaneously, subcutaneously, dermically, transdermically or intrathecally to the subject.

35. A method of treating pain in a subject comprising the administration of a nucleic acid sequence according to claim 23 or a vector comprising said nucleic acid sequence to a subject in need of treatment.


SEQUENCE LISTING (9 sequences)

SEQ ID NO: 1 - protein, 63 aa, synthetic construct
CFPGQVAGTTRAQPSCVEASIVIQKWWCHMNPCLEGEDCKVLPDYSGWSCSSGNKVKTTKVTR

SEQ ID NO: 2 - DNA, 189 bp, synthetic construct
  source 1..189: mol_type = other DNA
tgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcgg

SEQ ID NO: 3 - protein, 27 aa, synthetic construct
CFPGQVAGTTRAQPSCVEASIVIQKWW

SEQ ID NO: 4 - protein, 36 aa, synthetic construct
CHMNPCLEGEDCKVLPDYSGWSCSSGNKVKTTKVTR

SEQ ID NO: 5 - protein, 63 aa, synthetic construct
SFPGQVAGTTRAQPSSVEASIVIQKWWSHMNPSLEGEDSKVLPDYSGWSSSSGNKVKTTKVTR

SEQ ID NO: 6 - DNA, 189 bp, synthetic construct
  source 1..189: mol_type = other DNA
tcgtttccaggtcaagttgcgggaacaactcgtgcacaaccatcgtcggtagaggcctcaattgttatccaaaagtggtggtcgcacatgaacccctcgctcgaaggagaggactcgaaggtactgcctgactacagcgggtggtcatcgtcatcaggcaataaggtgaagacgaccaaagttacccgt

SEQ ID NO: 7 - protein, 76 aa, synthetic construct
HHHHHHENLYFQGCFPGQVAGTTRAQPSCVEASIVIQKWWCHMNPCLEGEDCKVLPDYSGWSCSSGNKVKTTKVTR

SEQ ID NO: 8 - protein, 76 aa, synthetic construct
HHHHHHENLYFQGSFPGQVAGTTRAQPSSVEASIVIQKWWSHMNPSLEGEDSKVLPDYSGWSSSSGNKVKTTKVTR

SEQ ID NO: 9 - DNA, 189 bp, synthetic construct
  source 1..189: mol_type = other DNA
tgctttccaggtcaagttgcgggaacaactcgtgcacaaccatcgtgcgtagaggcctcaattgttatccaaaagtggtggtgtcacatgaacccctgcctcgaaggagaggactgtaaggtactgcctgactacagcgggtggtcatgttcatcaggcaataaggtgaagacgaccaaagttacccgt